SEQUENCE LISTING (42 sequences)

SEQ ID NO: 1 - protein, 12 aa, Artificial Sequence
  Tub tag
SVEGEGEEEGEE

SEQ ID NO: 2 - protein, 4 aa, Artificial Sequence
  sortase A recognizing sequence
  MISC_FEATURE (3)..(3): Xaa can be any amino acid
LPXT

SEQ ID NO: 3 - protein, 6 aa, Artificial Sequence
  sortase A recognizing sequence
  MISC_FEATURE (3)..(3): Xaa may be any amino acid
  MISC_FEATURE (5)..(5): Xaa may be Gly or Ala
  MISC_FEATURE (6)..(6): Xaa may be any amino acid
LPXTXX

SEQ ID NO: 4 - protein, 8 aa, Artificial Sequence
  streptavidin-binding peptide
WSHPQFEK

SEQ ID NO: 5 - DNA, 458 bp, Artificial Sequence
  fusion protein
gcgctgaccccccaaagacacacgtgacccaccaccccatctctgaccatgaggccaccctgaggtgctgggccctgggtttctaccctgcggagatcacactgacctggcagcgggatggcgaggaccaaactcaggacactgagcttgtggagaccagaccagcaggagatagaaccttccagaagtgggcagctgtggtggtgccttctggagaagagcagagatacacatgccatgtacagcatgaggggctgccgaagcccctcaccctgagatgggagcctcctccgtctggatccagcgcttggtctcacccgcagttcgagaaaggtggaggttccggaggtggatcgggaggtggatcgtggagccacccgcagttcgaaaaagtggattcagtggagggcgagggagaggaagaaggcgaagaataataagcttgatccggctgctaa

SEQ ID NO: 6 - protein, 145 aa, Artificial Sequence
  fusion protein
RADPPKTHVTHHPISDHEATLRCWALGFYPAEITLTWQRDGEDQTQDTELVETRPAGDRTFQKWAAVVVPSGEEQRYTCHVQHEGLPKPLTLRWEPPPSGSSAWSHPQFEKGGGSGGGSGGGSWSHPQFEKVDSVEGEGEEEGEE

SEQ ID NO: 7 - DNA, 444 bp, Artificial Sequence
  fusion protein
gcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgggccatggtagctggagccacccgcagttcgaaaaagggggtggaagcggtggaggatcgggtagtggtagcgcttggagccacccgcagttcgaaaaactgcccgagaccggaggccaccaccatcatcaccattaataagcttgatccggctgctaacaaagcccgaaaggaagctgagttggctgctgccaccgctgagcaataactagcataaccccttggggcctctaaacgggtcttgag

SEQ ID NO: 8 - protein, 111 aa, Artificial Sequence
  fusion protein
AEITLTWQRDGEDQTQDTELVETRPAGDGTFQKWAAVVVPSGQEQRYTCHVQHEGLPKPLTLRWEPGHGSWSHPQFEKGGGSGGGSGSGSAWSHPQFEKLPETGGHHHHHH

SEQ ID NO: 9 - protein, 14 aa, Artificial Sequence
  Tub tag
VDSVEGEGEEEGEE

SEQ ID NO: 10 - protein, 6 aa, Artificial Sequence
  sortase A recognizing sequence
LPETGG

SEQ ID NO: 11 - protein, 7 aa, Artificial Sequence
  transglutaminase tag
PNPQLPF

SEQ ID NO: 12 - protein, 7 aa, Artificial Sequence
  transglutaminase tag
PKPQQFM

SEQ ID NO: 13 - protein, 6 aa, Artificial Sequence
  transglutaminase tag
GQQQLG

SEQ ID NO: 14 - protein, 5 aa, Artificial Sequence
  transglutaminase tag
RLQQP

SEQ ID NO: 15 - protein, 4 aa, Artificial Sequence
  transglutaminase tag
LLQA

SEQ ID NO: 16 - protein, 7 aa, Artificial Sequence
  transglutaminase tag
YAHQAHY

SEQ ID NO: 17 - protein, 7 aa, Artificial Sequence
  transglutaminase tag
YAHQPHY

SEQ ID NO: 18 - protein, 7 aa, Artificial Sequence
  transglutaminase tag
YPHQPHY

SEQ ID NO: 19 - protein, 7 aa, Artificial Sequence
  transglutaminase tag
YPHQAHY

SEQ ID NO: 20 - protein, 7 aa, Artificial Sequence
  transglutaminase tag
YSHQAHY

SEQ ID NO: 21 - protein, 7 aa, Artificial Sequence
  transglutaminase tag
YAHQAAH

SEQ ID NO: 22 - protein, 5 aa, Artificial Sequence
  transglutaminase tag
RLQQP

SEQ ID NO: 23 - protein, 5 aa, Artificial Sequence
  transglutaminase tag
RTQPA

SEQ ID NO: 24 - protein, 12 aa, Artificial Sequence
  transglutaminase tag
GGGYRYRQGGGG

SEQ ID NO: 25 - protein, 11 aa, Artificial Sequence
  transglutaminase tag
GGGYRYRQGGG

SEQ ID NO: 26 - protein, 5 aa, Artificial Sequence
  aldehyde-tag
  MISC_FEATURE (2)..(2): Xaa may be any amino acid
  MISC_FEATURE (4)..(4): Xaa may be any amino acid
CXPXR

SEQ ID NO: 27 - protein, 15 aa, Artificial Sequence
  avi tag
GLNDIFEAQKIEWHE

SEQ ID NO: 28 - protein, 13 aa, Artificial Sequence
  lipoic acid ligase tag
GFEIDKVWYDLDA

SEQ ID NO: 29 - protein, 10 aa, Artificial Sequence
  streptavidin-binding peptide
SAWSHPQFEK

SEQ ID NO: 30 - protein, 28 aa, Artificial Sequence
  di-tag3
WSHPQFEKGGGSGGGSGGGSWSHPQFEK

SEQ ID NO: 31 - protein, 23 aa, Artificial Sequence
  di-tag2
SHPQFEKGGGSGGGSWSHPQFEK

SEQ ID NO: 32 - protein, 28 aa, Artificial Sequence
  Twin-Strep-tag
WSHPQFEKGGGSGGGSGGSAWSHPQFEK

SEQ ID NO: 33 - protein, 4 aa, Artificial Sequence
  streptavidin analog fragment
VTAR

SEQ ID NO: 34 - protein, 4 aa, Artificial Sequence
  streptavidin analog fragment
IGAR

SEQ ID NO: 35 - protein, 8 aa, Artificial Sequence
  FLAG-tag
DYKDDDDK

SEQ ID NO: 36 - protein, 10 aa, Artificial Sequence
  Myc-tag
EQKLISEEDL

SEQ ID NO: 37 - protein, 9 aa, Artificial Sequence
  HA-tag
YPYDVPDYA

SEQ ID NO: 38 - protein, 11 aa, Artificial Sequence
  VSV-G-tag
YTDIEMNRLGK

SEQ ID NO: 39 - protein, 11 aa, Artificial Sequence
  HSV-tag
QPELAPEDPED

SEQ ID NO: 40 - protein, 14 aa, Artificial Sequence
  V5-tag
GKPIPNPLLGLDST

SEQ ID NO: 41 - protein, 15 aa, Artificial Sequence
  (G4S)3 linker
GGGGSGGGGSGGGGS

SEQ ID NO: 42 - protein, 294 aa, Artificial Sequence
  extracellular domain of chimeric antigen receptor
DIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELKGSTSGSGKPGSGEGSTKGEVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVSSWSHPQFEKGGGSGGGSGGSSAWSHPQFEKVDSVEGEGEEEGEE

(19) United States
(12) Patent Application Publication
(10) Pub. No.: US 2021/0246189 A1
(43) Pub. Date: Aug. 12, 2021

(54) A DOUBLE PEPTIDE TAG COMBINING REVERSIBILITY AND FLEXIBLE FUNCTIONALIZATION

(71) Applicants: TECHNISCHE UNIVERSITAET MUENCHEN, Munich (DE); LUDWIG-MAXIMILIANS-UNIVERSITAET MUENCHEN, Munich (DE)

(72) Inventors: Dirk BUSCH, Schliersee (DE); Manuel EFFENBERGER, Muenchen (DE); Heinrich LEONHARDT, Muenchen (DE); Andreas STENGL, Muenchen (DE)

(73) Assignees: TECHNISCHE UNIVERSITAET MUENCHEN, Munich (DE); LUDWIG-MAXIMILIANS-UNIVERSITAET MUENCHEN, Munich (DE)

(21) Appl. No.: 17/250,769

(22) PCT Filed: Aug. 30, 2019
(86) PCT No.: PCT/EP2019/073167

§ 371 Date: Mar. 2, 2021

(30) Foreign Application Priority Data
Sep. 3, 2018 (EP) 18192152.9

(51) Int. Cl. C07K 14/74; C12N 9/52; G01N 33/569; G01N 33/58

(52) U.S. Cl. CPC C07K 14/70539; C12N 9/52; C12Y 304/2207; C07K 2319/50; G01N 33/582; C07K 2319/22; C07K 2319/60; G01N 33/56977

(57) ABSTRACT

The present invention relates to a peptide comprising a reversible affinity tag (A); and a functionalization tag (F), wherein the peptide is linked to a target of interest (T). The peptide is useful as a versatile protein tag. The invention further provides structures comprising the peptide, nucleic acids, vectors, and host cells. Further, the invention provides methods of producing or using the peptide.

### CROSS-REFERENCE TO RELATED APPLICATIONS

The present application claims the right of priority of European patent application No. 18192152.9 filed with the European Patent Office on 3 Sep. 2018, the entire content of which is incorporated herein for all purposes.

### FIELD OF THE INVENTION

The present invention relates to a peptide comprising a reversible affinity tag (A); and a functionalization tag (F), wherein the peptide is linked to a target of interest (T). The peptide is useful as a versatile protein tag. The invention further provides structures comprising the peptide, nucleic acids, vectors, and host cells. Further, the invention provides methods of producing or using the peptide.

### BACKGROUND

A T cell's function is determined to a large part through the affinity of the T cell receptor (TCR) to the antigen presented on a major histocompatibility complex (MHC). Analysis of the interaction between a TCR and a peptide major histocompatibility complex (pMHC) has been challenging as the affinity of monomeric pMHC molecules is not strong enough for stable binding. Scaffolds allowing multimerization enable analyses of weak and transient interactions of molecules through the avidity gain of multivalent binding. Soluble biotinylated pMHC monomers can be multimerized on a dye-conjugated streptavidin backbone (‘tetramer’) (Altman, J. D. et al. Science 274, 94-96 (1996)). This enables sensitive detection and isolation of antigen-specific T cells and has opened up new avenues for in-depth T cell analysis in basic research and clinical immune monitoring (Busch, D. H. et al. J. Exp. Med. 188, 61-70 (1998)). The use of such non-reversible biotinylated pMHCs allows for analysis of antigen specific T cells independently from the differentiation state and have become the immunological standard technique ever since the method has been described for the first time. For clinical applications, it became possible to adoptively transfer T cells that have been identified with pMHC multimers to patients in order to restore lost or weakened immunity.

However, stable binding of pMHC tetramers can also deteriorate T cell in vivo functionality (O'Herrin, S. M. et al. J. Immunol. 167, 2555-2560 (2001); Maile, R. et al. J. Immunol. 167, 3708-14 (2001)). In addition, safety concerns arose, since pMHCs are infused to the patient together with the T cells.

Therefore, the fact that pMHC ligand binding to the TCR is only stable in its multivalent form has been exploited for the development of clinical cell selection and processing technologies (Knabel, M. et al. Nat. Med. 8, 631-7 (2002); Cobbold, M. et al. J. Exp. Med. 202, 379-386 (2005); Stemberger, C. et al. PLoS One 7, e35798 (2012); Neuenhahn, M. et al. Leukemia (2017). doi:10.1038/1eu.2017.16), and is further used for in-depth characterization of TCR:pMHC interactions. Reversible pMHC reagents—such as Strep-tagged ‘Streptamers’—allow the isolation of minimally manipulated cell products with no functional difference to cells that have never bound pMHC multimers (Knabel, M. et al. Nat. Med. 8, 631-7 (2002); Mohr, F. et al. Eur. J. Immunol. 1-26 (2017)). When reversible pMHC monomers themselves are labeled with a fluorophore, their dissociation from TCRs on living T cells can be tracked over time (Nauerth, M. et al. Sci. Transl. Med. 5, 192ra87 (2013); Hebeisen, M. et al. Cancer Res. (2015). doi:10.1158/0008-5472.CAN-14-3516). Through this, absolute and reproducible measurements of TCR k_{off}-rates can be achieved in a relatively easy and high-throughput compatible manner.

Until now, the versatility of pMHC reagents comes at the cost of distinct generation processes for each application (FIG. 1 A left). All three different types of the mentioned pMHC complexes have to be produced separately. They differ in their DNA sequence and consequently also in their protein sequence at the pMHC's C-terminus. Non-reversible, biotinylated pMHCs carry at their C-terminus a recognition sequence for the enzyme BirA. This enzyme promotes conjugation of one single biotin molecule per pMHC molecule, by which the non-reversible multimerization of the pMHCs with streptavidin is enabled (Altman, Science 1996). Reversible streptamers carry a C-terminal strep-tag sequence (Knabel et al. Nat. Med. 2002). This sequence enables a reversible multimerization with StrepTactin. Upon addition of biotin, this multimerization can be reversed, because biotin has a higher affinity than the strep-tag to StrepTactin. Fluorophore-conjugated streptamers for the determination of the structural avidity carry, further to the Strep-tag, an additional artificially introduced cysteine, via which a fluorophore can be covalently coupled to the streptamers by maleimide chemistry (Nauerth et al. Sci. Transl. Med. 2013).

Combination of all three pMHC complexes allow for an extensive T cell analysis, isolation, and characterization. Since the constructs—as described at the above—are different on the DNA level, they have to be encoded by different plasmids and have to be separately recombinantly expressed and purified. Eventually, they must also be separately folded to pMHC complexes (Busch et al. The Journal of Experimental Medicine 1996). Separate recombinant protein expression, in vitro refolding and pMHC purification are thereby laborious, time-consuming and prone to batch-to-batch variability. Ideally, the unique characteristics of those three constructs should emerge from one common pMHC precursor, allowing streamlining the generation process of pMHC reagents while simultaneously providing full flexibility to generate other pMHC multimer types with any function of interest. No approach has so far provided an all-in-one solution to produce versatile pMHC reagents within one streamlined generation process.

It is thus object of the invention to provide means and methods that at least partially overcomes the shortcomings of the current state of the art.

### SUMMARY OF THE INVENTION

The present invention relates to a peptide comprising (i) a reversible affinity tag (A); and (ii) a functionalization tag (F), wherein the peptide is linked to a target of interest (T), and (a) wherein the peptide and the target of interest have following configuration: T-A-F or F-A-T; or (b) wherein the peptide and the target of interest have following configuration: T-F-A or A-F-T, wherein the functionalization tag (F) is not a sortase A recognizing sequence or a tub tag.

The present invention also relates to a protein comprising the peptide of the invention.

The present invention also relates to a nucleic acid encoding a peptide of the invention or a protein of the invention.

The present invention also relates to a vector comprising a nucleic acid of the invention.

The present invention also relates to a host cell comprising a nucleic acid of the invention or a vector of the invention.

The present invention also relates to a method of producing a peptide of the invention or a protein of the invention comprising cultivation of the host cell of the invention under conditions allowing expression of the peptide or the protein.

The present invention also relates to a protein complex comprising a peptide of the invention or a protein or the invention and a multimerization reagent.

The present invention also relates to a method of determining the dissociation rate constant (k_{off}) of a specific binding partner and a target of interest, comprising detecting a first detectable label attached to the specific binding partner and a second detectable label attached to the target of interest, wherein the specific binding partner has been contacted with

(i) a first protein complex comprising at least one peptide of the invention comprising a first detectable label and a first multimerization reagent, wherein the protein complex is disruptable; and (ii) a second protein complex comprising at least one peptide of the invention that is conjugated to a biotin or biotin and a second multimerization reagent that is a streptavidin, avidin, streptavidin analog, or avidin analog that essentially irreversibly binds to a biotin or a biotin analog, wherein the at least one peptide of the second complex or the second multimerization reagent comprises a second detectable label that can be distinguished from the first detectable label.

The present invention also relates to a method of isolating a high-avidity T cell comprising (a) determining the dissociation rate constant (k_{off}) of a T cell in a sample obtained from a subject using the method of determining the dissociation rate constant (k_{off}) of the invention, and (b) isolating said T cell from a sample obtained from said subject.

The present invention also relates to the use of a peptide comprising (i) a reversible affinity tag; and a functionalization tag as a peptide tag.

### BRIEF DESCRIPTION OF THE DRAWINGS

FIG. 1

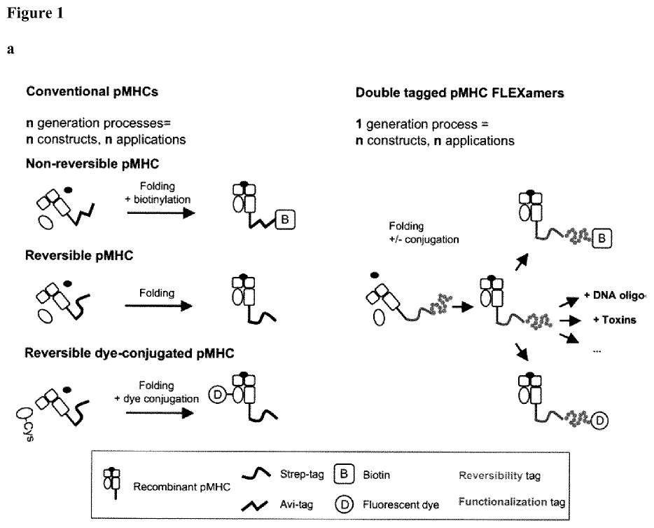

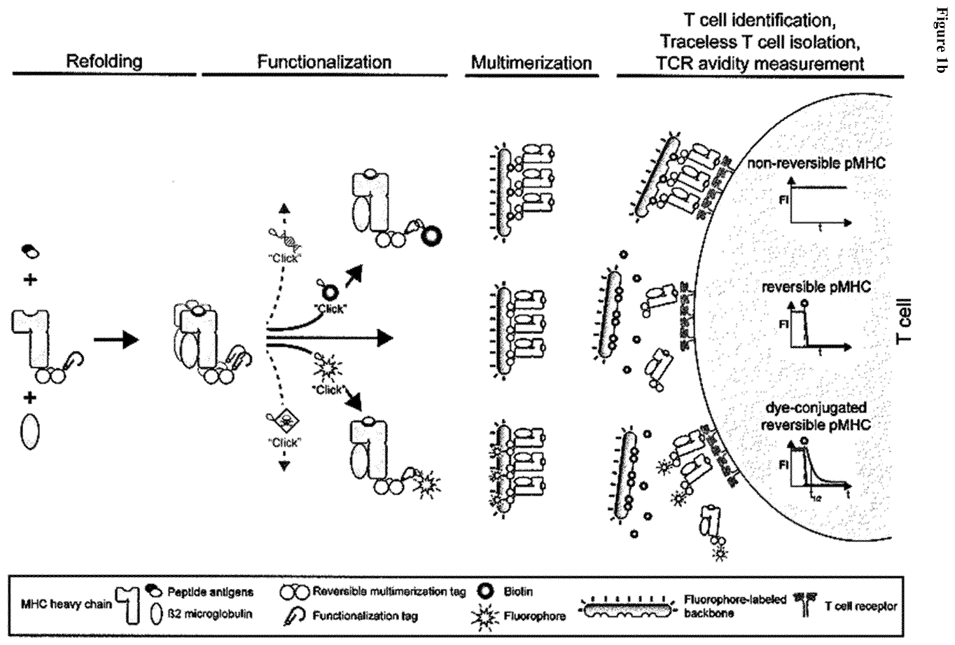

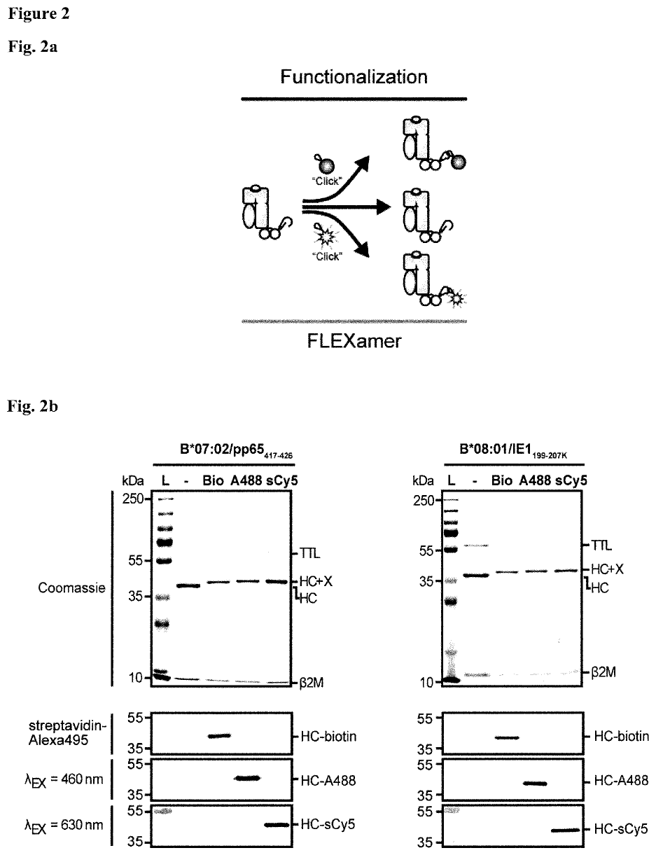

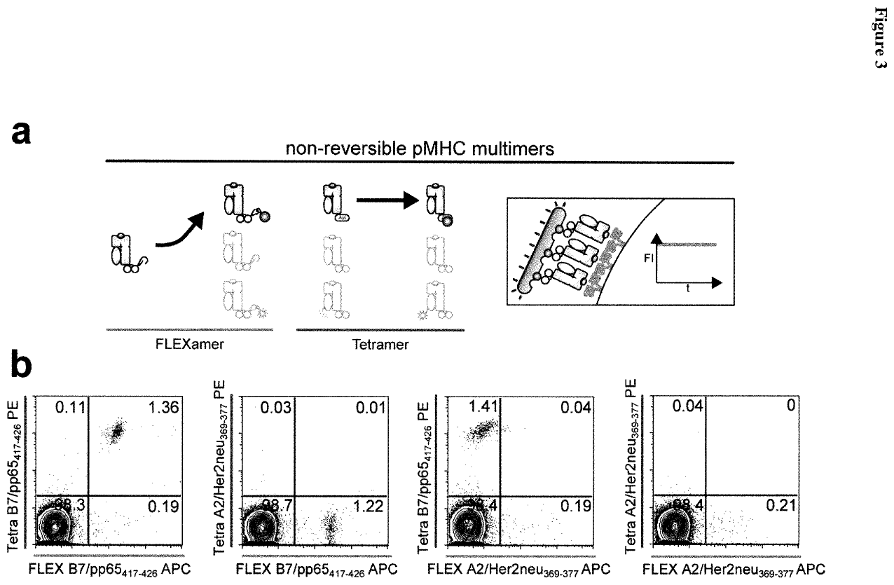

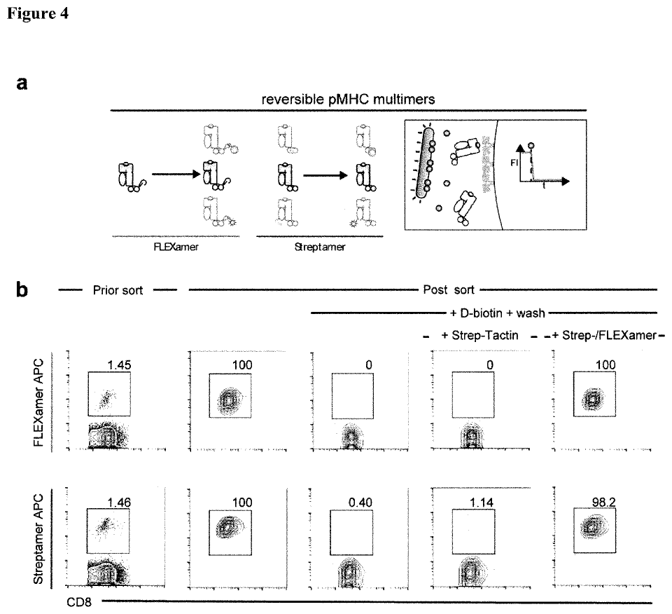

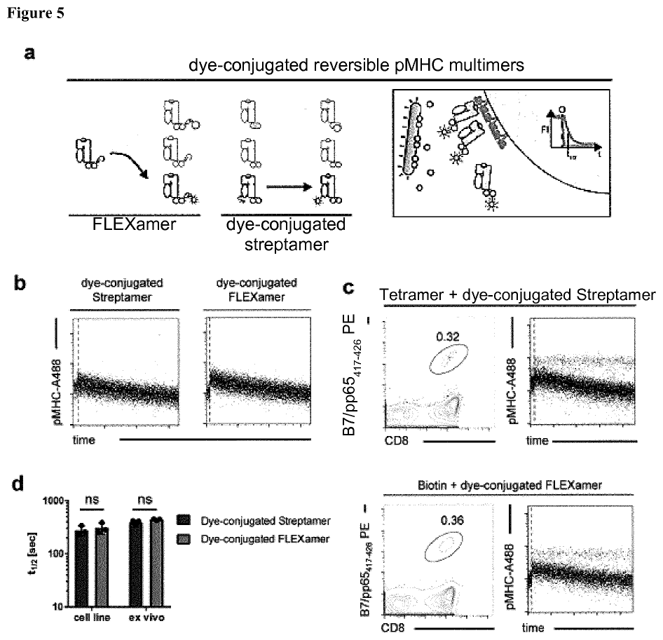

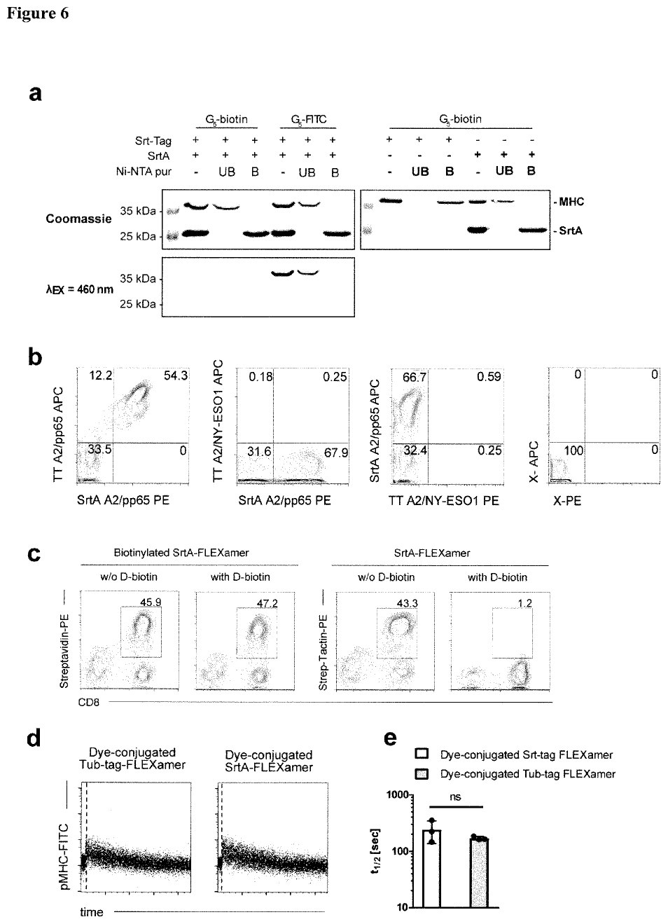

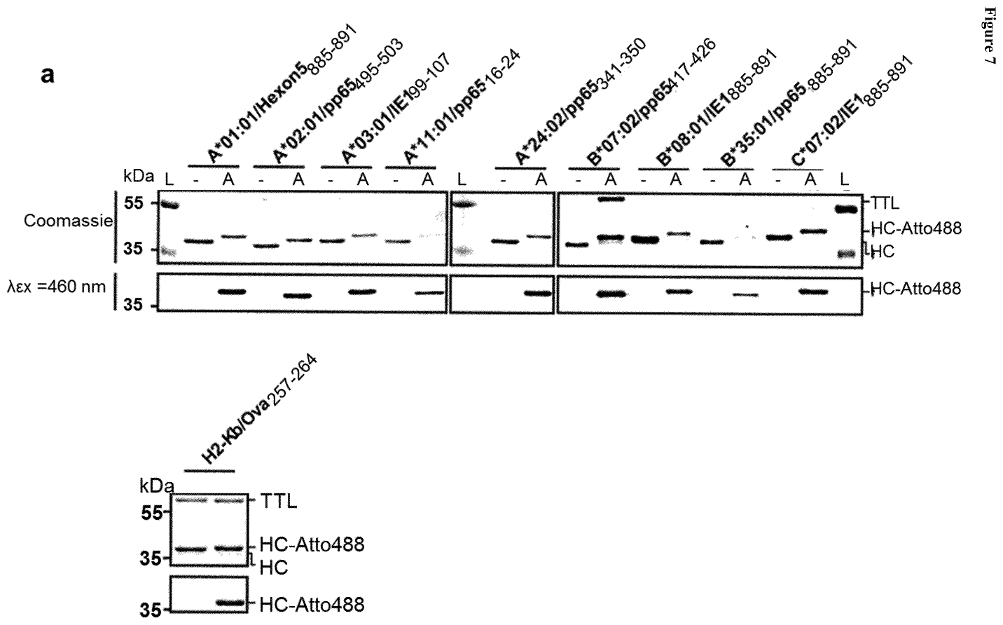

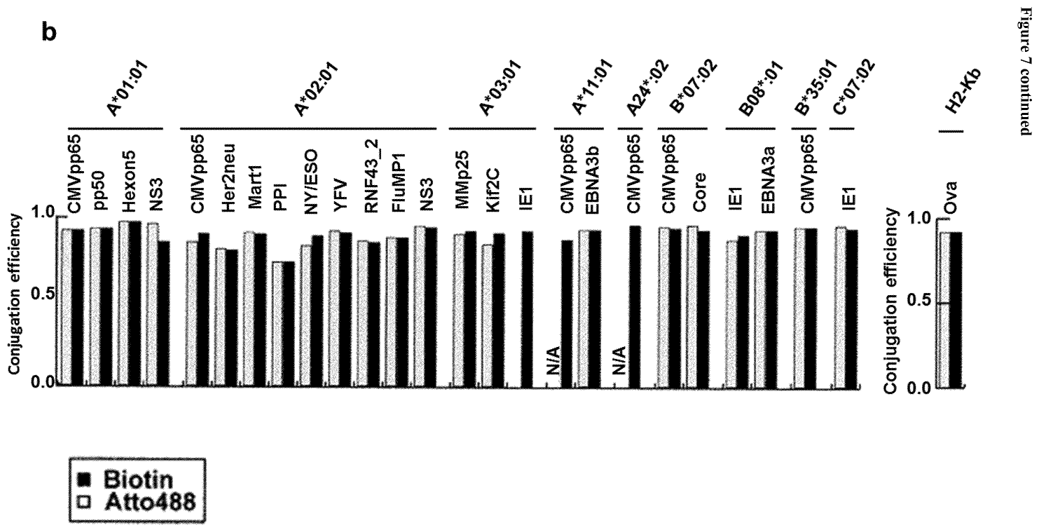

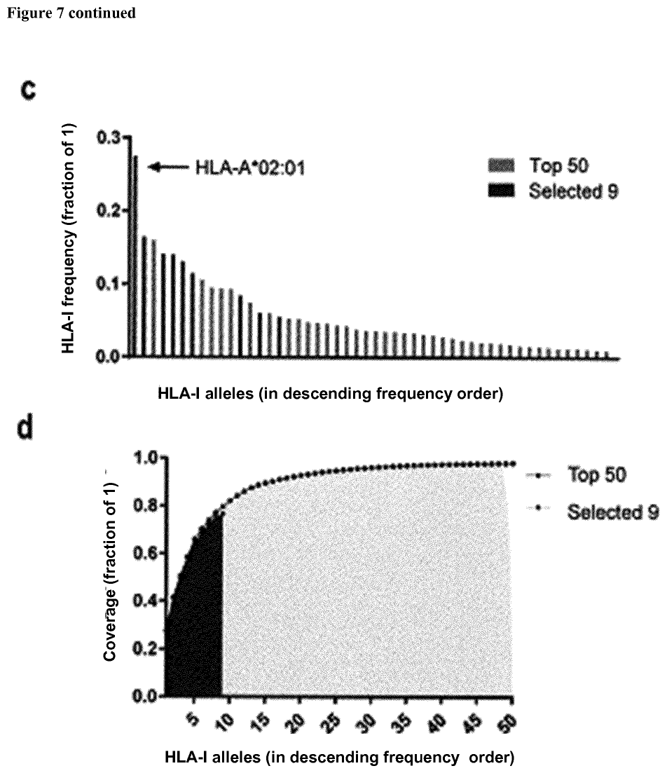

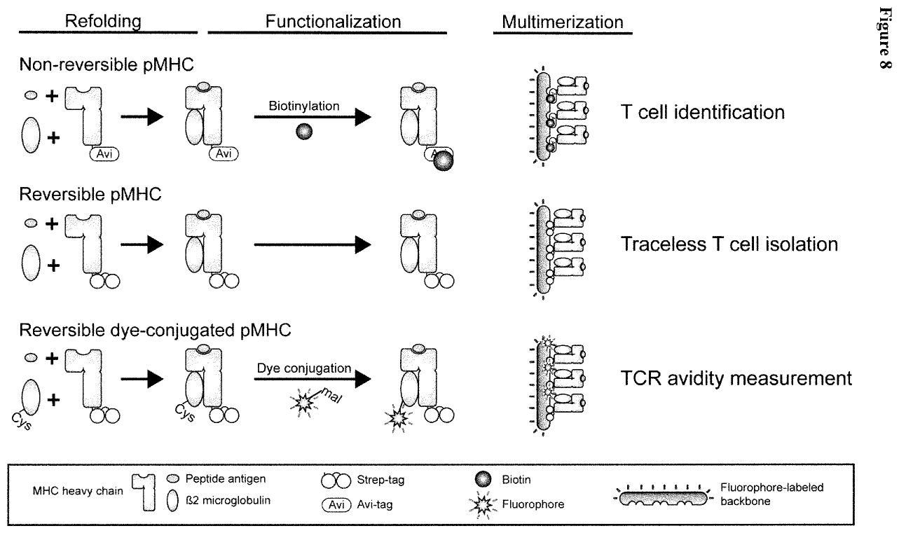

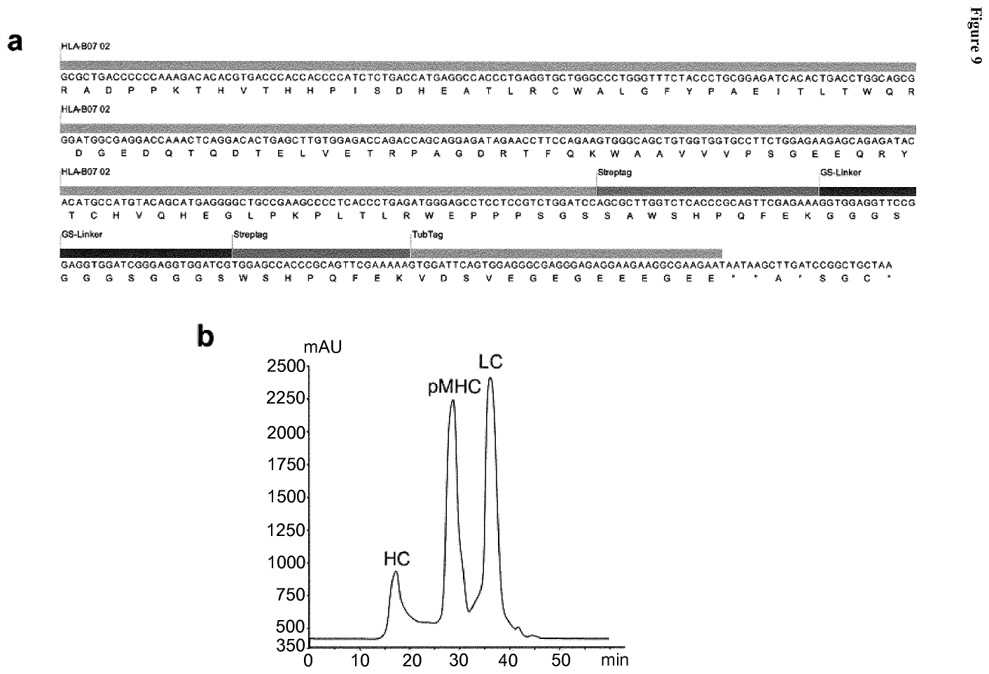

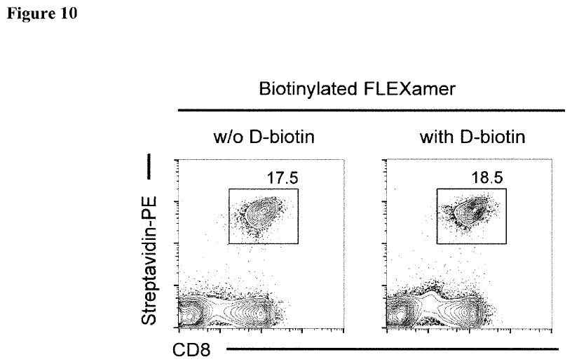

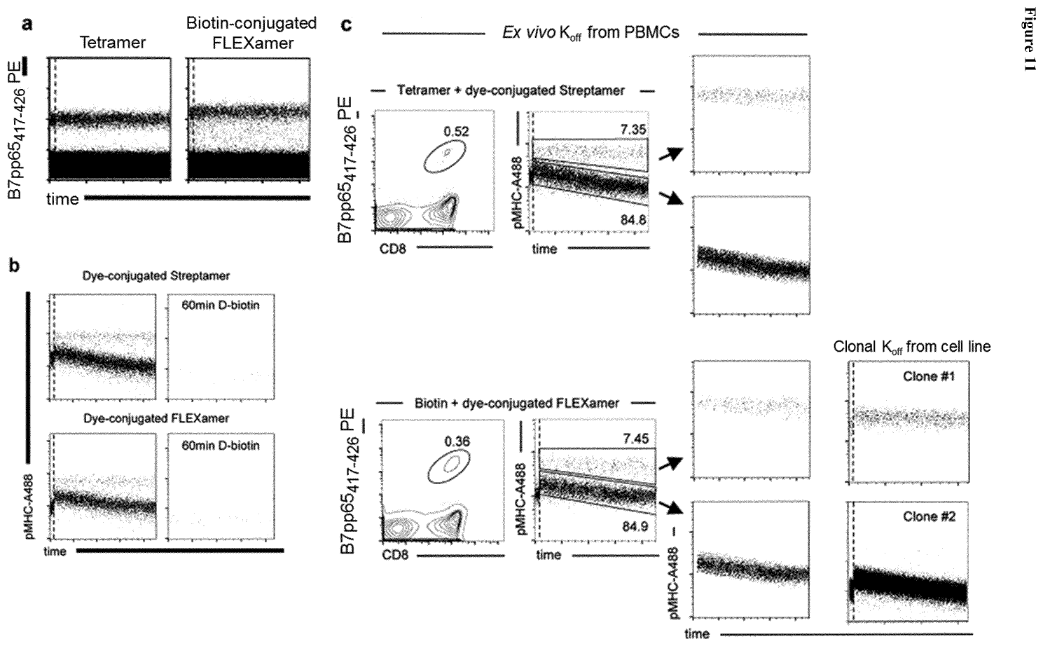

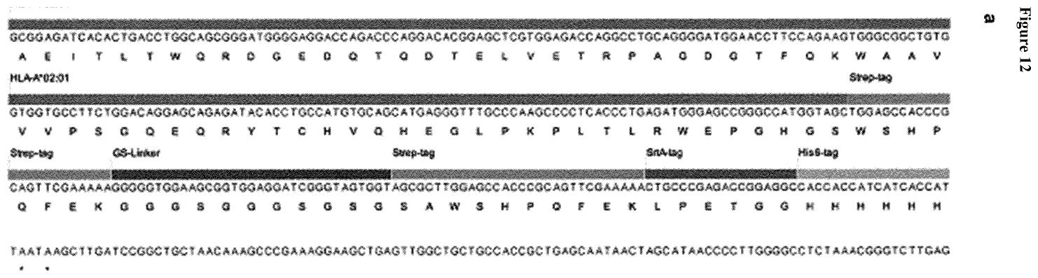

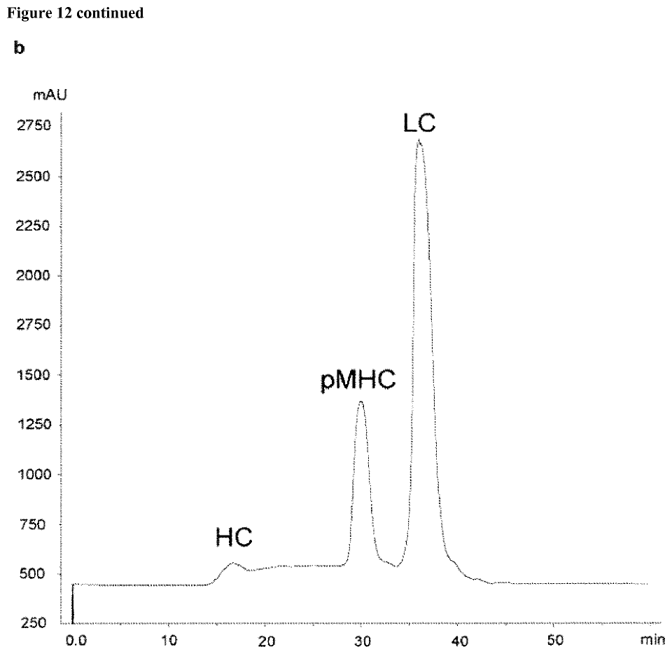

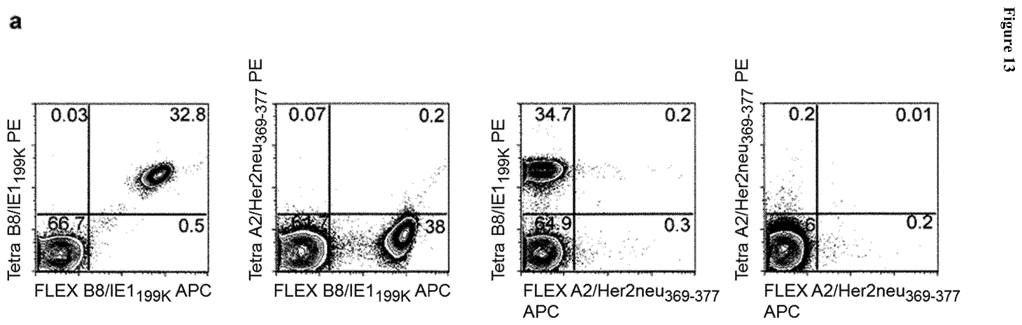

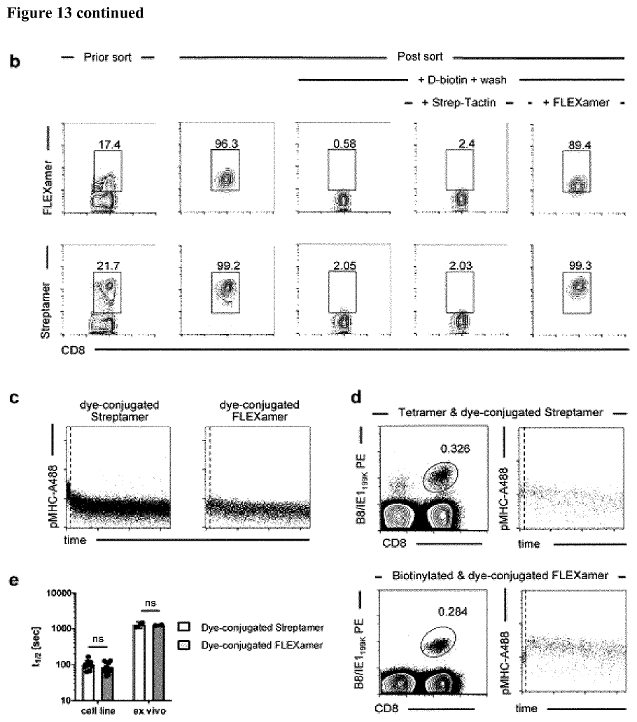

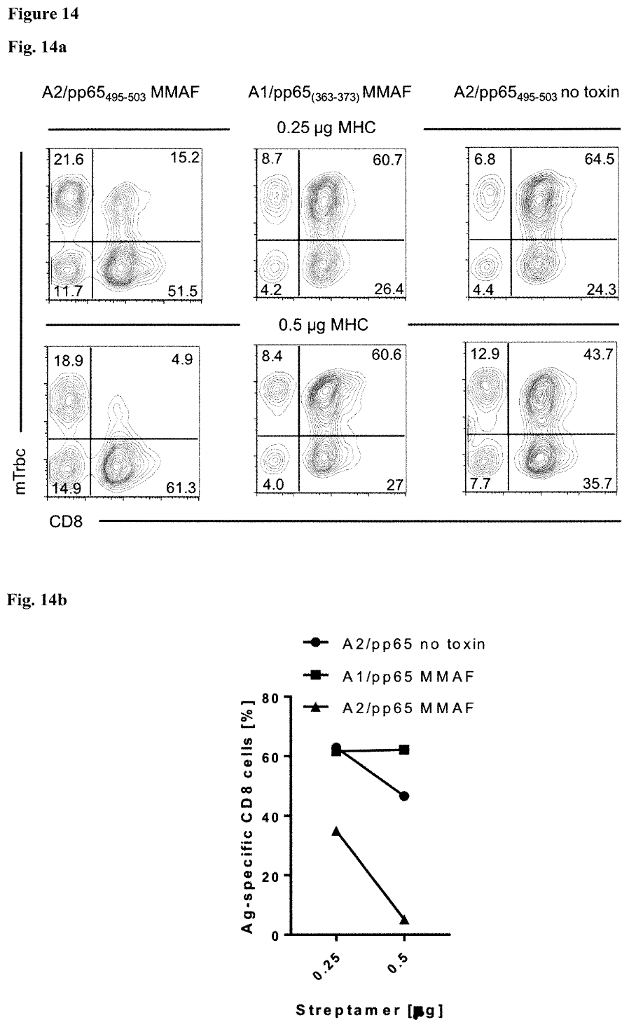

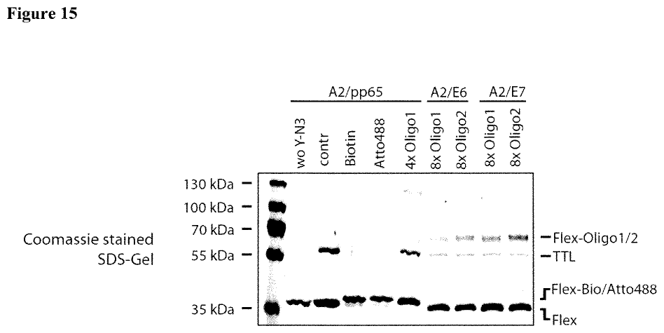

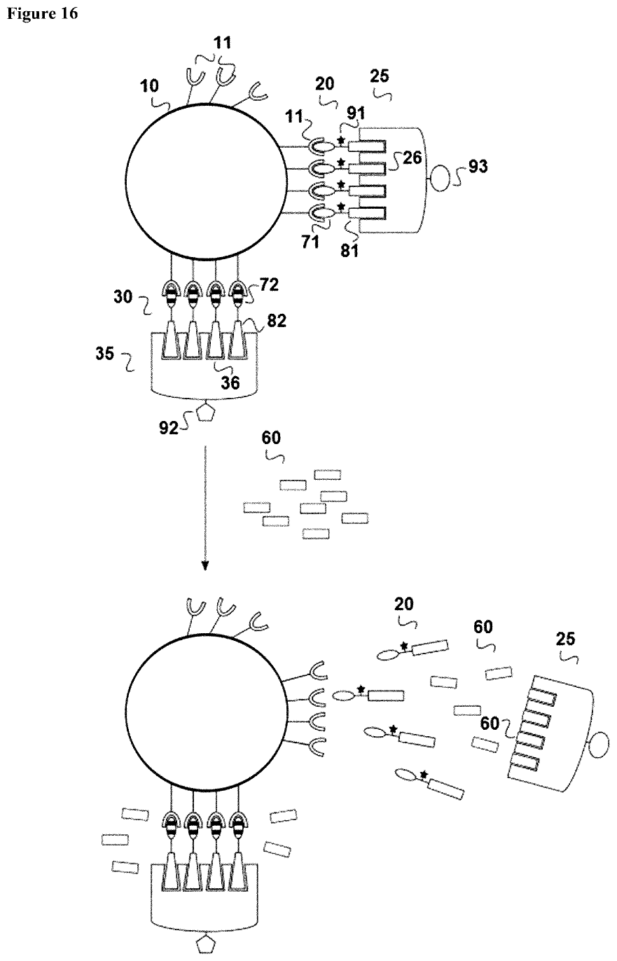

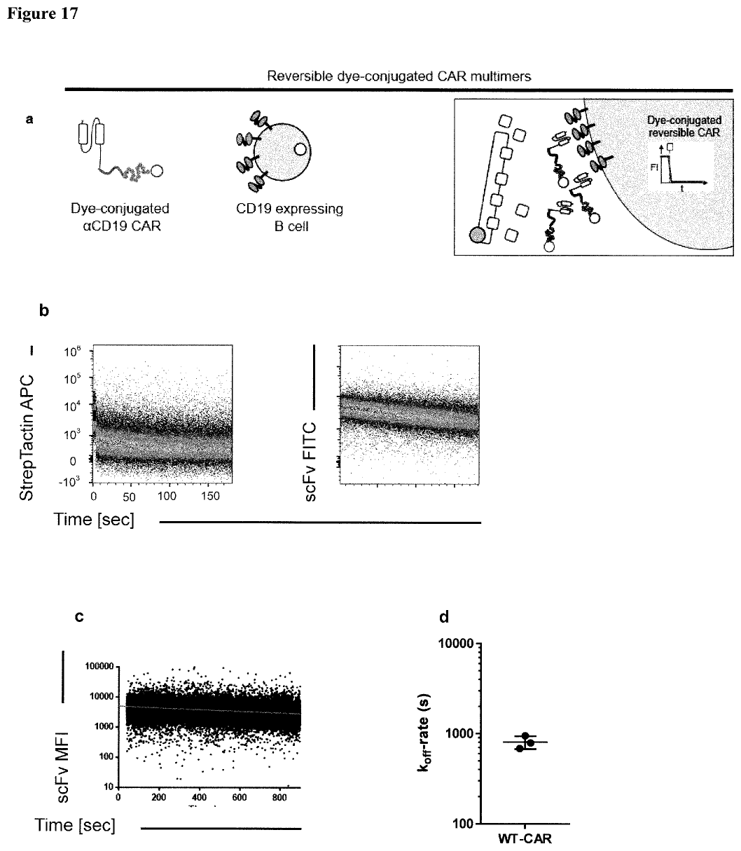

Double-tagged pMHC FLEXamers streamline generation of distinct pMHC reagents. (FIG. 1a) Schematic depiction of conventional pMHC reagent generation (left) versus pMHC generation from FLEXamers (right). Non-reversible pMHC reagents such as ‘tetramers’ are generated by folding of pMHC molecules that harbor an Avi-tag at the C-terminus of the heavy chain for site-specific biotinylation. Biotinylated pMHC monomers can be non-reversibly multimerized on streptavidin backbone. Reversible pMHCs carry affinity tags such as a Strep-tag at the C-terminus of the heavy chain that allow stable multimerization on Strep-Tactin backbone which can be reversed upon addition of the higher affine competitor molecule D-Biotin. Reversible dye-conjugated pMHC reagents can be generated through the introduction an additional artificial solvent exposed cysteine residue in the pMHC light chain which allows dye coupling via maleimide chemistry after folding (left). Folding of double-tagged pMHC ‘FLEXamers’ yields an already functional reversible pMHC monomer, which can be reversibly multimerized through a reversible affinity tag (reversibility tag). This pMHC can simultaneously serve as a precursor molecule to generate biotinylated pMHCs for tetramer generation, dye-coupled reversible pMHCs e.g. for koff-rate measurement or further pMHC reagents conjugated with any probe of interest via a site-specific functionalization tag (such as Tub- or the sortase A-tag) (right). (FIG. 1b) Schematic depiction of pMHC generation from ‘FLEXamers’ and their respective application in T cell immunology. pMHC FLEXamer complexes are assembled from combinations of double-tagged heavy chains, peptide antigens and B2 microglobulin. The double-tag consists of a reversible multimerization tag and a site-specific functionalization tag. This allows functionalization and multimerization of non-reversible, reversible or dye-conjugated reversible pMHCs from the same precursor molecule for T cell identification, traceless T cell isolation or TCR avidity measurement respectively.

FIG. 2

Efficient functionalization of double-tagged FLEXamers with biotin or fluorophores. (FIG. 2a) Schematic depiction of FLEXamer mediated functionalization into distinct pMHC reagents. (FIG. 2b) SDS-PAGE and western blot analysis of site-specific labeling of B*07:02 pp65 (417-426) and B*08:01 IE1 (199-207k) heavy chains by TTL-mediated incorporation of 3-azido-L-tyrosine (lane ‘-’) and subsequent click conjugation of DBCO-PEG4-biotin (Bio), DBCO-PEG4-Atto488 (A488) or DBCO-sulfoCy5 (sCy5). ‘HC+X’ indicates the molecular weight after conjugation of heavy chains (HC). Presence of the respective functional group was tested by Streptavidin-Alexa495 based detection of biotin or in-gel fluorescence of A488 and sCy5. Lane ‘L’ represents the molecular weight marker.

FIG. 3

T cell identification: comparing non-reversible biotinylated double-tagged FLEXamers versus conventional tetramers. (a) Schematic depiction comparing generation of non-reversible pMHC monomers using FLEXamer or BirA technique. (b) pMHC multimer staining of B7/pp65(417-426)-specific CD8+ T cells from peripheral blood of a CMV-seropositive donor. pMHCs were conventionally biotinylated (Tetra) and multimerized on streptavidin-PE or biotinylated via Tub-tag technique (FLEX) and multimerized on streptavidin-APC. Relevant epitope B7/pp65(417-426), irrelevant control epitope A2/Her2neu(369-377). Pregated on single, living CD8+ T cells.

FIG. 4

Traceless T cell isolation: comparing unmodified reversible double-tagged FLEXamers versus Streptamers. (a) Schematic depiction comparing generation of reversible pMHC monomers using FLEXamer or Streptamer technique. (b) Flow sort purification and re-staining of B7/pp65(417-426)-specific CD8+ T cells from peripheral blood of a CMV-seropositive donor with FLEXamers or conventional Streptamers. Pre-gated on single, living lymphocytes.

FIG. 5

TCR avidity measurement: comparing dye-conjugated double-tagged FLEXamers versus dye-conjugated Streptamers. (a) Schematic depiction comparing generation of dye-conjugated reversible pMHC monomers using FLEXamer technique or maleimid dye-chemistry on Streptamers. (b) Dissociation kinetics of a B7/pp65(417-426)-specific CD8+ T cell line measured with dye-conjugated Streptamers or dye-conjugated FLEXamers. Red dotted line indicates D-biotin addition. (c) Dissociation kinetics of B7/pp65(417-426)-specific CD8+ T cells from peripheral blood of a CMV-seropositive donor, stained with a combination of non-reversible biotinylated pMHCs and reversible dye-conjugated pMHCs. Non-reversible pMHC multimer+CD8+ T cells are gated for dissociation kinetic of dye-coupled pMHCs after D-biotin addition (red dotted line). (d) Quantification of technical triplicates of representative experiment shown in (b) and (c). One symbol represents one dissociation. Unpaired, nonparametric Kolmogorov-Smirnov test. Pre-gated on single, living CD8+ T cells in (b) and pre-gated on single, living lymphocytes in (c).

FIG. 6

Poly-functionality of double-tagged pMHCs can also be achieved by transpeptidation using SrtA-tag. (a) SDS PAGE of SrtA-mediated functionalization of Srt-tagged FLEXamer (MHC) with biotin or FITC. Transpeptidase reaction components are shown before (-) and after Ni-NTA purification (UB and B). The FLEXamers C-terminal His-tag is replaced with either G5-biotin or G5-FITC by the transpeptidation reaction. Labeled FLEXamers remain unbound to Ni-NTA-sepharose beads (UB) wherase the His-tagged SrtA is efficiently captured on the beads (B). Absence of FLEXamer in the bound fraction indicates efficient transpeptidation. Conjugation of FITC is further validated by in-gel fluorescence. Control reactions omitting either SrtA or the Srt-tag show no unspecific coupling. (b) Staining of PBMCs transduced with an A2/pp65(495-503)-specific TCR. pMHC reagents were multimerized from pMHC monomers either biotin-functionalized via Tub-tag technique or via SrtA-tag. Staining was performed with a combination of both pMHC reagents. Control stainings were performed with an irrelevant peptide epitope (A2/NY-ESO(157-165)). (c) PBMCs transduced with an A2/pp65(495-503)-specific TCR were stained with either biotinfunctionalized SrtA FLEXamers multimerized on streptavidin-PE or with their reversible SrtA FLEXamer precursor multimerized on Strep-Tactin-PE. Samples were incubated with or without D-biotin before acquisition. (d) Dissociation kinetic of PBMCs transduced with an A2/pp65(495-503)-specific TCR. Dye-conjugated pMHCs were generated either via Tub-tag technique or via SrtA-tag. Red dotted line indicates injection of D-biotin. (e) Quantification of technical triplicates of representative experiment shown in (d). One symbol represents one dissociation. Unpaired, non-parametric Kolmogorov-Smirnov test. Pre-gated on single, living CD8+ T cells in (b), pre-gated on single, living T cells in (d) and pre-gated on single, living CD8+mTrbc+ T cells in (d).

FIG. 7

Double-tagged FLEXamers allow highly efficient functionalization irrespective of HLA allotype and presented peptide epitope and can also be transferred to murine MHC. (a) SDS-PAGE analysis of site-specific labeling of HLA and MHC monomers by TTL. Incorporation of 3-azido-L-tyrosine (″) and subsequent click conjugation of DBCO-PEG4-Atto488. ‘HC+Atto488’ indicates the molecular weight after conjugation of heavy chains (HC). Presence of Atto488 was proven by in-gel fluorescence. ‘L’ indicates the molecular weight marker. (b) Conjugation efficacy of all 26 HLA-FLEXamers and murine FLEXamer H2-Kb/OVA coupled to Biotin or Atto488. (c) 50 most frequent HLA-class I alleles of EURCAU population in descending order with HC used for FLEXamer generation shown in red. (d) Cumulative coverage of 50 most frequent HLA-class I alleles (area under the curve in grey) or selected nine FLEXamer HC.

FIG. 8

Generation of conventional pMHC reagents. Non-reversible pMHC reagents (‘tetramers’) are generated by refolding of pMHC molecules harboring an Avi-tag at the C-terminus of the heavy chain for biotinylation. Biotinylated pMHC monomers can be non-reversibly multimerized on streptavidin. Reversible pMHCs carry an affinity-tag, e.g. a Strep-tag at the Cterminus of the heavy chain, that allows stable multimerization on Strep-Tactin, which can be reversed upon addition of higher affine competitor molecules. Reversible dye-conjugated pMHC reagents can be generated through the introduction of an artificial solventexposed cysteine in the pMHC light chain, which allows dye coupling via maleimide chemistry after folding.

FIG. 9

Strep- and Tub-double-tagged MHC heavy chains can be efficiently assembled into pMHC complexes. (a) Coding sequence of B*07:02 fused at the C-terminus to Strep-tag followed by Tub-tag sequence (SEQ ID NO: 05) and encoded amino acid sequence (SEQ ID NO: 06). (b) Representative size exclusion chromatogram profile of B*07:02 FLEXamer folding. Fractions of 2nd peak containing double tagged pMHC monomers were collected and pooled.

FIG. 10

Biotin-functionalized FLEXamers can be non-reversibly multimerized on a streptavidin backbone. Biotin functionalized FLEXamers were multimerized on streptavidin-PE and used to stain B7/pp65(417-426)-specific CD8+ T cells. Samples were incubated with or without D-biotin prior to acquisition. Pre-gated on single, living lymphocytes.

FIG. 11

Conventional tetramers and biotinylated FLEXamers allow continuous gating on antigen-specific CD8+ T cells over time also after addition of D-biotin, while dye-conjugated conventional Streptamers and dye-conjugated FLEXamers allow discrimination of antigen-specific CD8+ populations with distinct structural avidities. (a) Conventionally biotinylated pMHCs (tetramers upon multimerization) or biotin-functionalized FLEXamer pMHCs were multimerized on streptavidin-PE and used to stain B7/pp65(417-426)-specific CD8+ T cells from peripheral blood of a CMV-seropositive donor. The pMHC multimer signal is gated over time. Red dotted line indicates injection of D-biotin. (b) Dissociation kinetic of B7/pp65(417-426)-specific CD8+ T cells from peripheral blood of a CMV-seropositive donor. PBMCs were stained with reversible dye-conjugated pMHCs either conventionally generated via maleimid chemistry or using Tub-tag technique. Decay in fluorescence intensity directly after D-biotin incubation for 15 min and after 60 min for 2 min. Pre-gated on single, living, CD8+non-reversible pMHC+ T cells. (c) Dissociation kinetic of B7/pp65(417-426)-specific CD8+ T cells from peripheral blood of a CMV-seropositive donor shown in FIG. 1d. Manual gating on the population with the slower (upper) dissociation kinetic and the population with the faster (lower) kinetic. Subsequent gating on the kinetics is compared to dissociation kinetics derived from cell lines generated from the upper and lower population.

FIG. 12

Strep- and SrtA-double-tagged MHC heavy chains can be efficiently assembled into pMHC complexes. (a) Coding sequence of A*02:02 fused at the C-terminus to Strep-tag followed by Sortase A recognition tag (StrA-tag) sequence (SEQ ID NO: 07) and encoded amino acid sequence (SEQ ID NO: 08). (b) Representative profile of size exclusion chromatogram. Fractions of 2nd peak containing doubletagged pMHC monomers were collected and pooled.

FIG. 13

Double-tagged FLEXamer for distinct HLA epitope combination. (a) B8/IE1(199-207K)-specific CD8+ T cells from peripheral blood of a CMV-seropositive donor were stained with non-reversible pMHC multimers. pMHCs were either conventionally biotinylated (Tetra) and multimerized on streptavidin-PE or biotinylated via Tub-tag technique (FLEX) and multimerized on streptavidin APC. Cells were stained with either a combination of both reagents presenting the relevant epitope (B8/IE1(199-207K)), with combinations of relevant and irrelevant (A2/Her2neu(369-377)) epitope for each reagent, or with a combination of both reagents presenting the irrelevant epitope. (b) Flow cytometric sorting of B8/IE1(199-207K)-specific CD8+ T cells from peripheral blood of a CMV-seropositive donor using FLEXamers and Streptamers. FLEXamers and Streptamers were multimerized on Strep-Tactin-APC. Sort-purified population was subsequently split and either analyzed as purity control or incubated with D-biotin, with D-biotin and Strep-Tactin backbone only, or D-biotin and reversible pMHCs multimerized on Strep-Tactin backbone. (c) Dissociation kinetic of a B8/IE1(199-207K)-specific CD8+ T cell line measured with either dye-conjugated Streptamers or dye-conjugated FLEXamers. Red dotted line indicates time point of D-biotin addition. (d) Dissociation kinetic of B8/IE1(199-207K)-specific T cells in peripheral blood of a CMV-seropositive donor. PBMCs were stained with a combination of non-reversible biotinylated pMHCs and reversible dye-conjugated pMHCs. Reagents were generated either conventionally via BirA-mediated biotinylation and maleimid chemistry mediated dye coupling or using Tub-tag technique conjugating biotin or a dye. Non-reversible pMHC multimer+CD8+ population is gated for dissociation kinetic of dye-coupled pMHCs after addition of D-biotin (red dotted line) over time. (e) Quantification of technical replicates of representative experiments shown in (c) and (d). One symbol represents one dissociation. Unpaired, non-parametric Kolmogorov-Smirnov test. Pre-gated on single, living CD8+ T cells in (a) and (c), pre-gated on single, living lymphocytes in (b) and (d).

FIG. 14

Ag-specific killing of CD8 T cells using toxin armed Tub-tagged Streptamers. A) PBMCs transduced with an A2/pp65₍₄₉₅₋₅₀₃₎ specific TCR were cultured with 0.25 ug or 0.5 ug of either A2/pp65₄₉₅₋₅₀₃ pMHC functionalized with MMAF or Al/pp65₍₃₆₃₋₃₇₃₎ pMHC functionalized with MMAF or un-functionalized A2/pp65₄₉₅₋₅₀₃ pMHC for 5d. pMHCs were multimerized on StrepTacin backbone. After 5d culture, cells were stained with mAbs directed against CD8 and the murine constant region of the T cell receptor beta chain (mTrbc), which is only expressed on the TCR transgenic T cells. B) Quantification of plots shown in A). Mean of two biological duplicates.

FIG. 15

SDS-Gel analysis of flexamers labeled with DBCO-modified ssDNA Oligol or Oligo2.

FIG. 16

General principle of dissociation rate constant determination

FIG. 16 illustrates the principle of dissociation rate constant (k_{off}) determination using a first multimerization reagent (25) and a second multimerization reagent (35). The target cell (10) has bound to at least two receptor molecules R (11) the first receptor binding site B1 (71) of a first receptor binding reagent (20). The first receptor binding reagent (20) comprises a first receptor binding site B1 (71), a first detectable label (91) and a first binding partner C1 (81). At least two first receptor binding reagents (20) are reversibly bound to a first multimerization reagent (25) via the first binding partner C1 (81) comprised in the first receptor binding reagents (20) and the first binding site Z1 (26) comprised in the first multimerization reagent (25). The first multimerization reagent (25) optionally comprises a third detectable label (93). Furthermore, the target cell (10) has bound to at least two receptor molecules R (11) the second receptor binding site B2 (72) of a second receptor binding reagent (30). At least two second receptor binding reagents (30) are stably bound to a second multimerization reagent (35) via the second binding partner C1 (82) comprised in the second receptor binding reagents (30) and the second binding site Z2 (36) comprised in the second multimerization reagent (35). The second multimerization reagent (35) comprises a second detectable label (92). Alternatively, the second detectable label can be comprised in the second receptor binding agent (30) in addition to or instead of being comprised in the second multimerization reagent (not shown in the Figure). Upon contacting the complex with a competition reagent CR (60), the competition reagent CR (60) competes with the first binding partner C1 (81) comprised in the first receptor binding reagent (20) for the first binding site Z1 (26) comprised in the first multimerization reagent (25). Due to binding of the competition reagent CR (60) to the first binding site Z1 (26) comprised in the first multimerization reagent (25), the binding between the first receptor binding reagent (20) and the first multimerization reagent (25) is disrupted and the first multimerization reagent (25), and eventually the first receptor binding reagent (20), detach from the target cell (10).

FIG. 17

Chimeric antigen receptors tagged with Strep- and Tub-tag allow measurement of dissociation kinetics of CARs from living B cells.

(a) Schematic depiction of Strep- and Tub-tagged extracellular domain of chimeric antigen receptors functionalized with fluorescent dye and illustration of CAR dissociation from surface expressed CD19 on B cells. (b) Dissociation kinetic of fluorescently labeled CAR measured by flow cytometry. Decay in fluorescence intensity derived from StrepTactin backbone drops immediately after addition of D-biotin (left plot) while signal derived from fluorescent CAR decays slowly over the 30 min measurement. (c) One-phase exponential fit of CAR dissociation shown in (b) for determination of k_{off}-rates shown in (d). (d) Technical triplicates of koff-rates performed with fluorophore tagged CAR shown in (b) and (c). Pre-gated on single, living, CD20 positive B cells in (b).

### DETAILED DESCRIPTION

The inventors of the present application have surprisingly found that flexibility of pMHC production can be achieved by combining a functionalization tag with a reversible affinity tag, which will result in a peptide of the invention, which is also referred to herein as “double tag”. The double tag will unite reversibility of binding, which allows for the reversible multimerization of a tagged target structure of interest, with the opportunity to equip the target structure of interest with any desired additional functionality. In an illustrative example, the peptide of the invention may be conjugated to a pMHC (‘FLEXamer’), resulting in a tagged pMHC structure which can either reversibly multimerized or purified via the reversible affinity tag. Depending on the intended application, the tagged pMHC can be labeled with a biotin via the functionalization tag in order to enable irreversible multimerization. Alternatively, the tagged pMHC can be labeled with a detectable label, or a cytotoxic agent (FIG. 1a, right). This double tag can not only be applied in the production of pMHCs but can in principle be applied to any molecule of interest (target of interest), for which a versatile binding and/or functionalization may be desired.

Accordingly, the present invention relates to a peptide comprising (i) a reversible affinity tag (A); and (ii) a functionalization tag (F), wherein the peptide is linked to a target of interest (T).

The target of interest can be any molecule, for which a versatile binding and/or functionalization may be desired. Typically, said target of interest may be a protein, a peptide, a peptidomimetic, a nucleic acid, or a polysaccharide, just to name a few. Preferably, the target of interest is a protein. Of particular interest are target molecules that are capable of specifically binding to a certain structure. Non-limiting examples for such targets are a major histocompatibility complex (MHC), a T cell receptor or a structure comprising an extracellular domain thereof, an antibody or fragment thereof, a B cell receptor or a structure comprising an extracellular domain thereof, an aptamer or a structure comprising an extracellular domain thereof, a chimeric antigen receptor or a structure comprising an extracellular domain thereof, an integrin, or a proteinaceous binding molecule with antibody-like binding properties. In particular targets that have low or moderate affinity may benefit from multimerization, e.g. through the reversible affinity tag, due to avidity effects.

A major histocompatibility complex (MHC) is a cell surface protein essential for the acquired immune system to recognize foreign molecules in vertebrates, which in turn determines histocompatibility. The main function of MHC molecules is to bind to antigens derived from pathogens and display them on the cell surface for recognition by the appropriate T-cells. MHC complexes are divided into three subgroups: class I, class II, and class III. The MHC according to the invention may be selected from any one of these classes. Preferably, the MHC according to the invention is of class I or II, most preferably of class I. As used herein, the term “MHC” may also include a MHC molecule that is conjugated with a peptide, a so-called peptide major histocompatibility complex (pMHC). The term “MHC” may also include a derivative of an MHC, such as a single chain MHC.

A T-cell receptor (TCR) is a molecule found on the surface of T cells, or T lymphocytes, that is responsible for recognizing fragments of antigen as peptides bound to major histocompatibility complex (MHC) molecules. The TCR is composed of two different protein chains, which may be an alpha (a) and a beta ((3) chain or a gamma (γ) and a delta (6) chain. As used herein, the term “TCR” may also include structures that are derived from a TCR, such as a single chain TCR.

The term “antibody” generally refers to a proteinaceous binding molecule with immunoglobulin-like functions. Typical examples of an antibody are immunoglobulins, as well as derivatives or functional fragments thereof which still retain the binding specificity. Techniques for the production of antibodies are well known in the art. The term “antibody” also includes immunoglobulins (Ig's) of different classes (i.e. IgA, IgG, IgM, IgD, IgE, IgY etc.) and subclasses (such as IgG1, IgG2 etc.), even if recombinantly produced in foreign hosts using techniques known to those skilled in the arts. Illustrative examples of an antibody are full length immunoglobulins, F_{ab} fragments, F(ab′)₂, Fᵥ fragments, single-chain Fᵥ fragments (scFᵥ), diabodies or domain antibodies (Holt L J et al., Trends Biotechnol. 21(11), 2003, 484-490). Domain antibodies may be single domain antibodies, single variable domain antibodies or immunoglobulin single variable domain having only one variable domain, which may be VH or VL, that specifically bind an antigen or epitope independently of other V regions or domains. The definition of the term “antibody” thus also includes embodiments such as chimeric, single chain and humanized antibodies. The term “antibody” may also include fragments of antibodies. The fragment is preferably an antigen-binding fragment, which means that the fragment may at least comprise a heavy chain variable region and a light chain variable region of an antibody. Examples for a divalent antibody fragment comprise, but are not limited to divalent antibody fragment is an (Fab)₂′-fragment, or a divalent single-chain Fv fragment. Examples of monovalent antibody fragments include, but are not limited to an Fab fragment, an Fv fragment, a single domain antibody, and a single-chain Fv fragment (scFv).

The term “chimeric antigen receptor” or “CAR” or “CARs” as used herein refers to engineered receptors, which graft an antigen specificity onto a receptor of a cytotoxic cell, for example T cells, NK cells and macrophages. A CAR may typically comprise at least one antigen specific targeting region (ectodomain), a transmembrane domain, and an intracellular signaling domain (endodomain). After the antigen specific targeting region binds specifically to a target antigen, the intracellular signaling domain activates intracellular signaling. For example, the intracellular signaling domain can redirect T cell specificity and reactivity toward a selected target in a non-MHC-restricted manner, exploiting the antigen-binding properties of the antigen specific targeting region. The non-MHC-restricted antigen recognition gives T cells expressing the CAR the ability to recognize an antigen independent of antigen processing. A typical example for the ectodomain is an scFv fragment or a CD19 ligand. A typical example for the transmembrane domain is a CD28 transmembrane domain. A typical example of an endodomain is CD3-zeta.

A B-cell receptor or “BCR” a used herein refers to an immunoglobulin molecule that form a type 1 transmembrane receptor protein usually located on the outer surface of a lymphocyte type known as B cells. The B-cell receptor includes both CD79 and the immunoglobulin. The receptor's binding moiety is composed of a membrane-bound antibody. The B cell receptor extends both outside the cell and inside the cell. An extracellular domain of the B cell receptor typically comprises the variable domain of an immunoglobulin heavy chain and/or the variable domain of an immunoglobulin light chain.

The term “aptamer” as used herein refers to an oligonucleotide that is capable of forming a complex with an intended target substance. The complexation is target-specific in the sense that other materials which may accompany the target do not complex to the aptamer. It is recognized that complexation and affinity are a matter of degree; however, in this context, “target-specific” means that the aptamer binds to target with a much higher degree of affinity than it binds to contaminating materials. The meaning of specificity in this context is thus similar to the meaning of specificity as applied to antibodies, for example. The aptamer may be prepared by any known method, including synthetic, recombinant, and purification methods, and may be used alone or in combination with other aptamers specific for the same target.

Examples of proteinaceous binding molecules with antibody-like binding properties that can be used as receptor binding reagent that specifically binds the receptor molecule include, but are not limited to, an aptamer, a mutein based on a polypeptide of the lipocalin family, a glubody, a protein based on the ankyrin scaffold, a protein based on the crystalline scaffold, an adnectin, an avimer, a EGF-like domain, a Kringle-domain, a fibronectin type I domain, a fibronectin type II domain, a fibronectin type III domain, a PAN domain, a G1a domain, a SRCR domain, a Kunitz/Bovine pancreatic trypsin Inhibitor domain, tendamistat, a Kazal-type serine protease inhibitor domain, a Trefoil (P-type) domain, a von Willebrand factor type C domain, an Anaphylatoxin-like domain, a CUB domain, a thyroglobulin type I repeat, LDL-receptor class A domain, a Sushi domain, a Link domain, a Thrombospondin type I domain, an immunoglobulin domain or a an immunoglobulin-like domain (for example, domain antibodies or camel heavy chain antibodies), a C-type lectin domain, a MAM domain, a von Willebrand factor type A domain, a Somatomedin B domain, a WAP-type four disulfide core domain, a F5/8 type C domain, a Hemopexin domain, an SH2 domain, an SH3 domain, a Laminin-type EGF-like domain, a C2 domain, “Kappabodies” (Ill. et al. “Design and construction of a hybrid immunoglobulin domain with properties of both heavy and light chain variable regions” Protein Eng 10:949-57 (1997)), “Minibodies” (Martin et al. “The affinity-selection of a minibody polypeptide inhibitor of human interleukin-6” EMBO J 13:5303-9 (1994)), “Janusins” (Traunecker et al. “Bispecific single chain molecules (Janusins) target cytotoxic lymphocytes on HIV infected cells” EMBO J 10:3655-3659 (1991) and Traunecker et al. “Janusin: new molecular design for bispecific reagents” Int J Cancer Suppl 7:51-52 (1992), a nanobody, a adnectin, a tetranectin, a microbody, an affilin, an affibody or an ankyrin, a crystallin, a knottin, ubiquitin, a zinc-finger protein, an autofluorescent protein, an ankyrin or ankyrin repeat protein or a leucine-rich repeat protein, an avimer (Silverman, Lu Q, Bakker A, To W, Duguay A, Alba B M, Smith R, Rivas A, Li P, Le H, Whitehorn E, Moore K W, Swimmer C, Perlroth V, Vogt M, Kolkman J, Stemmer W P 2005, Nat Biotech, December; 23(12):1556-61, E-Publication in Nat Biotech. 2005 Nov. 20 edition); as well as multivalent avimer proteins evolved by exon shuffling of a family of human receptor domains as also described in Silverman J, Lu Q, Bakker A, To W, Duguay A, Alba B M, Smith R, Rivas A, Li P, Le H, Whitehorn E, Moore K W, Swimmer C, Perlroth V, Vogt M, Kolkman J, Stemmer W P, Nat Biotech, December; 23(12):1556-61, E-Publication in Nat. Biotechnology. 2005 Nov. 20 edition).

A “functionalization tag” as used herein is a peptide (sequence) that is specifically recognized by an enzyme, which is preferably capable of catalyzing the conjugation of a molecule of interest to the molecule carrying this functionalization tag.

In one embodiment, the functionalization tag is based on a short hydrophilic, unstructured sequence recognized by tubulin tyrosine ligase (TTL), which is also referred to herein as “Tub-tag” (Schumacher, D. et al. Angew. Chemie—Int. Ed. 54, 13787-13791 (2015); Prota, A. E. et al. J. Cell Biol. 200, 259-70 (2013)). Preferably, the sequence recognized by TTL comprises Val-Asp-Ser-Val-Glu-Gly-Glu-Gly-Glu-Glu-Glu-Gly-Glu-Glu (SEQ ID NO: 09). The sequence recognized by TTL may also comprise the sequence Ser-Val-Glu-Gly-Glu-Gly-Glu-Glu-Glu-Gly-Glu-Glu (SEQ ID NO: 01). Functionalization is based on TTL that is naturally involved in the intracellular regulation of microtubule stability. TTL recognizes a TTL recognition motif at the C-terminus of alphatubulin and posttranslationally attaches a terminal tyrosine residue. When recombinantly fused to a molecule of interest, the recognition motif (Tub-tag) allows the TTL-mediated attachment of an unnatural tyrosine derivative that carry uniquely reactive groups for chemoselective conjugation such as strain-promoted alkyne azide cycloadditions (Schumacher, D. et al. Angew. Chemie—Int. Ed. 54, 13787-13791 (2015); Schumacher et al., J Clin Immunol (2016) 36 (Suppl 1):S100-S107). TTL-catalyzed attachment of tyrosine derivatives, such as 3-azido-L-tyrosine, allows subsequent addition of a variety of functional groups, such as biotin or dyes, by highly efficient and mild click chemistry. Compared to other chemo-enzymatic approaches the TTL reaction is not reversible, the product does not suffer from hydrolysis and the substrate tyrosine derivatives represent easy-to-synthesize chemical compounds. It is understood that the tubulin tyrosine ligase recognizing sequence is preferably at the C terminal end of the double tag and/or the target of interest, since functionalization of the Tub-tag occurs at the C terminus of the recognition sequence.

In one embodiment, the functionalization tag is a sortase A recognition sequence. The sortase A recognizing sequence may comprise the sequence Leu-Pro-Xaa1-Thr, wherein Xaa1 is any amino acid (SEQ ID NO: 02). Preferably, the sequence comprises the sequence Leu-Pro-Xaa1-Thr-Xaa2-Xaa3, wherein Xaa1 is any amino acid, Xaa2 is Gly or Ala, and Xaa3 is any amino acid (SEQ ID NO: 03). Preferably, the sequence comprises the sequence Leu-Pro-Glu-Thr-Gly-Gly (SEQ ID NO: 10). Functionalization is based on the transpeptidase Sortase A (Staphylococcus aureus), which has intensively been studied for the site-specific modification of proteins and antibodies (Mao H et al. J Am Chem Soc.2004; 126:2670-1, Popp M W et al. Nat Chem Biol. 2007; 3:707-8; Mohlmann S, et al. Chembiochem. 2011; 12:1774-80). The enzyme attacks the amide bond following the threonine recognition sequence LPXT (SEQ ID NO: 02). This lead to the release of the terminal amino acid and formation of a thioacyl intermediate. A following nucleophilic attack of another peptide equipped with a cargo of choice results in the reformation of an amide-bond and thereby the site-specific functionalization of the protein. It is understood that the sortase A recognizing sequence is preferably at the C terminal end of the double tag and/or the target of interest, since amino acids that are C terminal of the threonine comprised in the recognition sequence will be released.

In one embodiment, the functionalization tag is a transglutaminase recognition sequence, also referred to as “transglutaminase tag”. Such a transglutaminase tag may have a sequences selected from the group consisting of PNPQLPF (SEQ ID NO: 11), PKPQQFM (SEQ ID NO: 12), GQQQLG (SEQ ID NO: 13), RLQQP (SEQ ID NO: 14), and LLQA (SEQ ID NO: 15) (van Vught et al Comput Struct Biotechnol J. 2014; 9: e201402001; Schumacher et al., J Clin Immunol (2016) 36 (Suppl 1):S100-S107), or selected from the group consisting of YAHQAHY (SEQ ID NO: 16), YAHQPHY (SEQ ID NO: 17), YPHQPHY (SEQ ID NO: 18); YPHQAHY (SEQ ID NO: 19); YSHQAHY (SEQ ID NO: 20); YAHQAAH (SEQ ID NO: 21); RLQQP (SEQ ID NO: 22); RTQPA (SEQ ID NO: 23), GGGYRYRQGGGG (SEQ ID NO: 24), GGGYRYRQGGG (SEQ ID NO: 25) (Oteng-Pabi S K, Clouthier C M, Keillor J W (2018) Design of a glutamine substrate tag enabling protein labelling mediated by Bacillus subtilis transglutaminase. PLoS ONE 13(5): e0197956. https://doi.org/10.1371/journal.pone.0197956).

In one embodiment, the functionalization tag is a formylglycine generating enzyme (FGE) recognition sequence, also referred to herein as “aldehyde-tag”. Such a recognition sequence may comprise the sequence CXPXR, where X can be any amino acid (SEQ ID NO: 26), but is preferably serine, threonine, alanine, or glycine. FGE oxidizes the cysteine side chain of the peptide sequence CXPXR (SEQ ID NO: 26) to a formylglycine. Oxime forming reactions or, to gain hydrolytically stable products, Pictet-Spengler type reactions are used to conjugate payload to this bioorthogonal group (Dierks T et al. Cell. 2005; 121:541-52; Carrico I S et al. Nat Chem Biol. 2007; 3:321-2; Agarwal P et al. Proc Natl Acad Sci. 2013; 110:46-51; Agarwal P et al. Bioconjug Chem. 2013; 24:846-51).

In one embodiment, the functionalization tag is an Avi-Tag (GLNDIFEAQKIEWHE, SEQ ID NO: 27), which is a peptide allowing biotinylation by the enzyme BirA. Such a biotinylated structure may e.g. be isolated by streptavidin.

In one embodiment, the functionalization tag is a lipoic acid ligase recognition sequence, also referred to herein as “lipoic acid ligase tag”. Such a lipoic acid ligase tag may have the sequence GFEIDKVWYDLDA (SEQ ID NO: 28). Functionalization is conducted via a two-step labeling procedure using lipoic acid ligase A. In a first step, a p-iodophenyl carboxylic acid may be coupled to the amino group of the lysine side chain comprised in the lipoic acid ligase tag by lipoic acid ligase A (W37V) as a chemical handle which may afterward be specifically labeled with an ethynyle-modified cargo by palladium catalyzed Sonogashira cross-coupling (Hauke et al., Bioconjugate Chem. 2014, 25, 1632-1637).

The reversible affinity tag according to the invention may be any amino acid sequence that can reversibly bind a given binding partner. A typical application for an affinity tags is in protein purification, where the tag is appended to a protein of interest. The protein of interest may then be specifically bound and isolated by specific binding partners of the affinity tag, which are typically conjugated to a carrier, followed by disrupting the bond between affinity tag and specific binding partner, which results in the release of the protein of interest. Multiple reversible affinity tags are known to the person skilled in the art. Some exemplary reversible affinity tags are for example described in Kimple et al., Curr Protoc Protein Sci 2013 Sep. 24; 73:Unit 9.9. doi: 10.1002/0471140864.ps0909s73.

A preferred affinity tag according to the invention is an oligohistidine tag. An oligohistidine tag, sometimes also referred to as “polyhistidine tag” or “His-Tag” consists of 2-10, typically 6 consecutive histidine residues. The oligohistidine tag binds divalent metal atoms, such as Ni²⁺, Co²⁺, Cu²⁺ or Zn²⁺, that may be conjugated to a carrier. Binding between the oligohistidine tag and the carrier can be disrupted by contacting the complex with a competition reagent, such as imidazole or histidine. Alternatively, these binding complexes can be disrupted by metal ion chelation, e.g. by addition of EDTA or EGTA. In the context of the present invention, the binding partner of the oligohistidine tag may be a nickel or cobalt-based magnetic bead, as e.g. described by Tischer S et al. Int Immunol. 2012 September; 24(9):561-72.

Another preferred affinity tag according to the invention is a streptavidin or avidin binding peptide. Said streptavidin or avidin binding peptide may comprise the sequence Trp-Ser-His-Pro-Gln-Phe-Glu-Lys (SEQ ID NO: 04) or Ser-Ala-Trp-Ser-His-Pro-Gln-Phe-Glu-Lys (SEQ ID NO: 29). The streptavidin or avidin binding peptide might, for example, be a single peptide such as the “Strep-Tag®” described in U.S. Pat. No. 5,506,121, for example, or a streptavidin binding peptide having a sequential arrangement of two or more individual binding modules as described in International Patent Publication WO 02/077018. Examples of streptavidin binding peptides having a sequential arrangement of two or more individual binding modules include the di-tag3 sequence (WSHPQFEKGGGSGGGSGGGSWSHPQFEK; SEQ ID NO: 30), the di-tag2 sequence Trp-Ser-His-Pro-Gln-Phe-Glu-Lys-(GlyGlyGlySer)₂-Trp-Ser-His-Pro-Gln-Phe-Glu-Lys (SEQ ID NO: 31) that are described in International Patent Application WO02/077018 or U.S. Pat. No. 7,981,632 or the sequence WSHPQFEKGGGSGGGSGGSAWSHPQFEK (SEQ ID NO: 32, also known as Twin-Strep-Tag®). The streptavidin binding peptide may be bound by a streptavidin, or avidin, or a streptavidin analog, or an avidin analog that reversibly binds to said streptavidin or avidin binding peptide; or an streptavidin analog having the amino acid sequence Val⁴⁴-Thr⁴⁵-Ala⁴⁶-Arg⁴⁷ (SEQ ID NO: 33) at positions 44 to 47 of the wild-type streptavidin sequence or the streptavidin analog having the amino acid sequence Ile⁴⁴-Gly⁴⁵-Ala⁴⁶-Arg⁴⁷ (SEQ ID NO: 34) at positions 44 to 47 of the wild-type streptavidin sequence. Both these muteins are described in U.S. Pat. No. 6,103,493, for example, and are commercially available under the trademark Strep-Tactin® from IBA GmbH, Gottingen, Germany. Here, when contacting the complex of the streptavidin binding peptide and the streptavidin, or avidin, or a streptavidin analog, or an avidin analog with excess free biotin, which in this context acts a competition reagent, the streptavidin-binding peptide is displaced from the streptavidin, or avidin, or a streptavidin analog, or an avidin analog, thereby disrupting the bond.

The reversible affinity may also comprise an antigen that can be bound by an antibody or antibody fragment against said antigen. The antigen may, for example, be an epitope tag. Examples of suitable epitope tags include, but are not limited to, FLAG-tag (sequence: DYKDDDDK, SEQ ID NO: 35), Myc-tag (sequence: EQKLISEEDL, SEQ ID NO: 36), HA-tag (sequence: YPYDVPDYA, SEQ ID NO: 37), VSV-G-tag (sequence: YTDIEMNRLGK, SEQ ID NO: 38), HSV-tag (sequence: QPELAPEDPED, SEQ ID NO: 39), and V5-tag (sequence: GKPIPNPLLGLDST, SEQ ID NO: 40). The antigen may also be a protein, for example, maltose binding protein (MBP), chitin binding protein (CBP) or thioredoxin as an antigen. In these cases, the complex formed between the antigen and the antibody can be disrupted by adding the free antigen as competition reagent, i.e. the free peptide such as a Myc-tag or the HA-tag (epitope tag) or the free protein (such as MBP or CBP). In this context, it is noted that in case the FLAG-tag is used as reversible affinity tag and the antibody or antibody fragment binds to the FLAG tag, it is e.g. possible of disrupting this reversible bond by addition of the free FLAG peptide.

The invention also encompasses that the reversible affinity tag may comprise glutathione S-transferase (GST) which may bind to a glutathione that is conjugated to a carrier. Here, the bond between the GST and glutathione can be dissociated by addition of excess glutathione as competition reagent. The free glutathione may competitively displace the glutathione conjugated to a carrier that is bound to GST. The reversible affinity tag may further be a calmodulin binding peptide.

The invention also encompasses that the reversible affinity tag may comprise an immunoglobulin Fc portion which may be bound by a protein selected from the group consisting of protein A, protein G, protein a/g, and protein L. The bond between the immunoglobulin Fc portion and the protein A, protein G, protein a/g, or protein L may e.g. be disrupted by applying an acidic pH.

According to the invention, the peptide comprising the reversible affinity tag (A) and functionalization tag (F), and the target of interest (T) may have following configurations (where applicable, the configuration is given in the direction from N terminus to C terminus): T-A-F, T-F-A, F-A-T, or A-F-T.

Accordingly, the reversible affinity tag (A) may be located between the target of interest (T) and the functionalization tag (F). Alternatively, the functionalization tag (F) may be located between the target of interest (T) and the reversible affinity tag (A). If the functionalization tag (F) is located between the target of interest (T) and the reversible affinity tag (A), i.e. if the construct has the configuration T-F-A or A-F-T, it will preferably be envisioned that the functionalization tag (F) is not a sortase A recognition sequence, since the transpeptidase reaction will result in the loss of the structure unit that is C terminal of said sortase A recognition sequence. Similarly, if the functionalization tag (F) is located between the target of interest (T) and the reversible affinity tag (A), i.e. if the construct has the configuration T-F-A or A-F-T, it will preferably be envisioned that the functionalization tag (F) is not a Tub-tag sequence, since functionalization of the Tub-tag requires a free C terminus. For similar reasons, it may be envisioned by the invention that the functionalization tag (F) is not a sortase A recognition sequence or Tub-tag sequence if the target of interest (T) and the peptide has the configuration F-A-T.

According to the invention, it is understood that the reversible affinity tag and the functionalization tag may be located on the same polypeptide chain. Both tags may be directly fused together, i.e. the reversible affinity tag and the functionalization tag may be directly adjacent to other. Alternatively, both tags may be separated by a linker. Such a linker is preferably a polypeptide linker that is part of the polypeptide chain comprising the reversible affinity tag and the functionalization tag. A preferred linker is a peptide linker. Accordingly, said linker may comprise one or more amino acids, such as 1, 2, 3, 4, 5, 6, 7, 8, 9, 10, 11, 12, 13, 14, 15, 16, 17, 18, 19, 20 or more amino acids. Preferred peptide linkers are described herein, including glycine-serine (GS) linkers, glycosylated GS linkers, and proline-alanine-serine polymer (PAS) linkers. A preferred linkers includes, a (G₄S)₃ as described in SEQ ID NO: 41.

According to the invention, a preferred target of interest is a protein. In such a case, it is preferred that the protein or at least a subunit of the protein is located in the same polypeptide chain as the reversible affinity tag and the functionalization tag. The protein target of interest and the functionalization tag may be directly at adjacent to each other, but may also be linked together via a linker. Such a linker is preferably a polypeptide linker that is part of the polypeptide chain comprising the reversible affinity tag and the functionalization tag. A preferred linker is a peptide linker. Accordingly, said linker may comprise one or more amino acids, such as 1, 2, 3, 4, 5, 6, 7, 8, 9, 10, 11, 12, 13, 14, 15, 16, 17, 18, 19, 20 or more amino acids. Preferred peptide linkers are described herein, including glycine-serine (GS) linkers, glycosylated GS linkers, and proline-alanine-serine polymer (PAS) linkers. A preferred linkers includes, a (G₄S)₃ as described in SEQ ID NO: 41.

As an illustrative example, where the target of interest is an MHC or comprises at least a domain of an MHC or at least a polypeptide chain of an MHC, the peptide comprising the reversible affinity tag and the functionalization tag may be fused to at least one domain of the MHC or the at least one polypeptide chain of the MHC. Here, the reversible affinity tag may be fused to the C terminus of the alpha chain of the MHC while the functionalization tag is fused to the C terminus of the reversible affinity tag. Here, the reversible affinity tag may be a Tub tag or a sortase A recognition sequence, while the functionalization tag may be Strep-tag or Strep-tag comprising sequence or an oligohistidine tag. As another illustrative example, where the target of interest is an MHC, the reversible affinity tag may be fused to the C terminus of the beta chain of the MHC while the functionalization tag is fused to the C terminus of the reversible affinity tag. Here, it may be desirable to include a peptide linker between the C terminus of the MHC beta chain and the reversible affinity tag. Again, the reversible affinity tag may be a Tub tag or a sortase A recognition sequence, while the functionalization tag may be Strep-tag or Strep-tag comprising sequence or an oligohistidine tag.

Similarly, where the target of interest is a T cell receptor, an antibody, a B cell receptor, or any other structures comprising more than one polypeptide strand, the peptide comprising the reversible affinity tag and the functionalization tag can be fused to either one of the polypeptide chains comprised in the target of interest, either directly or via a linker.

The target of interest and the peptide comprising the reversible affinity tag and the functionalization tag may also be linked to each other by means other than fusion of polypeptide chains. However, it is desired that the target of interest and the peptide are covalently linked. For example, the target of interest and the peptide may be linked through chemical conjugation. This can e.g. achieved by linking the target of interest and the peptide via an amino acid side chain comprising a reactive group for conjugating. Such a reactive group may be a thiol group, such as a thiol group comprised in a cysteine, which can be conjugated to another structure via maleimide-mediated methodologies. For the purpose of conjugation, artificial amino acids may be introduced to the amino acid sequence of either the target of interest or the peptide. Generally, such artificial amino acids are designed to be more reactive and thus to facilitate the conjugation to the desired compound. Such artificial amino acids may be introduced by mutagenesis, for example, using an artificial tRNA is para-acetyl-phenylalanine. Methods for conjugating the target of interest and the peptide comprising the first and the functionalization tag are familiar to the skilled person. Some of the methods are e.g. described by Koniev, O. et al. (2015) Chem. Soc. Rev. 44 (15): 5495-5551 or Francis, M. B. et al. (2010) Current Opinion in Chemical Biology. 14 (6): 771-773.

According to the invention, the functionalization tag can be conjugated to another compound. In principle, this other component can be any component of interest, e.g. a label (moiety). Such a label may for example be a detectable label.

In general, such a “detectable label” may be any appropriate chemical substance or enzyme, which directly or indirectly generates a detectable compound or signal in a chemical, physical, optical, or enzymatic reaction. For example, a fluorescent or radioactive label can be conjugated to the functionalization tag to generate fluorescence or x-rays as detectable signal. Alkaline phosphatase, horseradish peroxidase and β-galactosidase are examples of enzyme labels (and at the same time optical labels) which catalyze the formation of chromogenic reaction products. In a preferred embodiment, the detectable label refers to detectable entities that can be used for the detection of the target of interest in flow cytometry. Preferably, the label does not negatively affect the characteristics of the target of interest. Examples of labels are fluorescent labels such as phycoerythrin, allophycocyanin (APC), Brilliant Violet 421, Alexa Fluor 488, coumarin or rhodamines to name only a few. The label may be a direct label, i.e. a label that is bound the functionalization tag, for example, covalently coupled (conjugated) to the functionalization tag. Alternatively, the label may be an indirect label, i.e. a label which is bound to a further reagent which in turn is capable of binding or being conjugated to the functionalization tag. The detectable label may further be a nucleic acid, such as an oligonucleotide having a recognition sequence. Such a recognition sequence may be a random sequence. This random sequence may be barcode sequence that has been incorporated into the nucleic acid molecules and can be used to identify the target molecule that has been conjugated with said nucleic acid.

Another preferred label may be a biotin label. A biotin label may be desirable, since biotin may essentially irreversibly bind to streptavidin, avidin, a streptavidin analog, or an avidin analog. As described herein, versatility of the double peptide tag of the invention is one of the inventive concepts of the present invention. This versatility may include the versatility of producing constructs that, depending on the functionalization, bind to streptavidin, avidin, a streptavidin analog, or an avidin analog in either a reversible or essentially irreversible manner. This can e.g. be achieved by incorporating a peptide tag that comprises a functionalization tag and a reversible affinity tag that is a streptavidin or avidin binding peptide. In the absence of a biotinylation, the peptide will reversibly bind to streptavidin, avidin, a streptavidin analog, or an avidin analog via the streptavidin or avidin binding peptide. However, the peptide can also be conjugated to a biotin via the functionalization tag. In such a case, the peptide will essentially irreversibly bind to streptavidin, avidin, a streptavidin analog, or an avidin analog via the biotin. Accordingly, the peptide comprising the reversible affinity tag and the functionalization tag will provide the versatility that reversibility of binding to streptavidin, avidin, a streptavidin analog, or an avidin analog can be easily modulated on the final complex comprising the target of interest, the reversible affinity tag and the functionalization tag.

Another label may be a toxin. As used herein, “toxin” means any agent that is detrimental to cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracinedione, mitoxantrone, mithramycin, actinomycin D, 1-dehydrotestosterone, glucocorticoids, procaine, tetracaine, lidocaine, propranolol, and puromycin and analogs or homologs thereof. Other toxins include, for example, ricin, CC-1065 and analogues, the duocarmycins. Still other toxins include diptheria toxin, and snake venom (e.g., cobra venom). Further toxins may comprise a radioactive agent including any radioisotope that is effective in staining or destroying a cell. Examples include, but are not limited to, indium-111, cobalt-60. Additionally, naturally occurring radioactive elements such as uranium, radium, and thorium, which typically represent mixtures of radioisotopes, are suitable examples of a radioactive agent. The metal ions are typically chelated with an organic chelating moiety. When conjugating the peptide to a toxin, the target of interest can be used to deliver the toxin to a predefined location, such as a malignant cell, depending on the binding property and/or specificity of the target of interest.

If the target of interest conjugated to the double tag of the invention has the configuration T-A-F, and if the functionalization tag is a sortase A recognizing sequence, it may be desirable to attach a further affinity tag C terminal of the sortase A recognition sequence, which allows for an easier purification of the complex. Such a further affinity tag need not be the same tag as the first reversible affinity tag. Any affinity tag that is commonly used for protein purification may be used as a further affinity tag. Suitable affinity tags are described herein. For example, the further affinity tag can be an oligohistidine tag. Since a successful conjugation of the functionalization tag using sortase A will result in the loss of the C-terminal further affinity tag, unconjugated molecules or peptides comprising the further affinity tag that have been cleaved off can be easily removed using an affinity agent for the further affinity tag, such as an immobilized affinity agent. If oligohistidine is the further affinity tag, the affinity agent may be a divalent metal cation know to bind oligohistidine, such as Ni²⁺, Co²⁺, Cu²⁺ or Zn²⁺, which may be immobilized on a carrier comprising NTA.

The present invention also relates to a protein comprising the target of interest, the reversible affinity tag and the functionalization tag as disclosed herein. The protein may consist of one polypeptide chain comprising the target of interest, the reversible affinity tag, and the functionalization tag. The protein may also consist of two or more polypeptide chains. In such a case, the protein comprises at least one polypeptide chain of the target of interest that is fused to the reversible affinity tag and the functionalization tag. A preferred protein of the invention comprises an MHC, preferably a pMHC.

The present invention also relates to a nucleic acid molecule comprising a sequence encoding a peptide of the invention as described herein or a protein of the invention is described herein. The nucleic acid molecule may be a DNA or an RNA molecule. The nucleic acid molecules of the invention may be part of a vector or any other kind of cloning or expression vehicle, such as a plasmid, a phagemid, a phage, a baculovirus, a cosmid or an artificial chromosome. The nucleic acid molecule, may allow expression of the peptide or protein. It may include sequence elements that contain information regarding to transcriptional and/or translational regulation, and such sequences may be “operably linked” to the nucleotide sequence encoding the protein. An operable linkage is a linkage in which the regulatory sequence elements and the sequence to be expressed are connected in a way that enables gene expression. The precise nature of the regulatory regions necessary for gene expression may vary among species, but in general these regions include a promoter, which, in prokaryotes, contains both the promoter per se, i.e., DNA elements directing the initiation of transcription, as well as DNA elements which, when transcribed into RNA, will signal the initiation of translation. Such promoter regions normally include 5′ non-coding sequences involved in initiation of transcription and translation, such as the −35/−10 boxes and the Shine-Dalgarno element in prokaryotes or the TATA box, CAAT sequences, and 5′-capping elements in eukaryotes. These regions can also include enhancer or repressor elements as well as translated signal and leader sequences for targeting the native protein to a specific compartment of a host cell.

Such a vehicle described herein may include, aside from the regulatory sequences described herein and a nucleic acid sequence encoding a peptide or protein described herein, replication and control sequences derived from a species compatible with a host cell that is used for expression as well as selection markers conferring a selectable phenotype on transformed or transfected cells. Large numbers of suitable cloning vectors are known in the art and are commercially available. Accordingly, the present invention also relates to a vector comprising the nucleic acid molecule of the invention.

Cloning or expression of nucleic acid molecule or the vector of the invention can be conducted at least partially in vivo, using host cells transformed with the nucleic acid or vector, or to which the nucleic acid molecule or vector has been transferred by other means including transduction or transfection. Transfer of DNA can be performed using standard techniques. Thus, the disclosure is also directed to a host cell containing a nucleic acid molecule or a vector as disclosed herein.

The invention further relates to a method of production of a peptide of the invention, a protein of the invention, or a complex of a target of interest and a peptide of the invention. The peptide or protein of the invention may be produced starting from the nucleic acid coding for the peptide or protein. The method can be carried out in vivo, wherein the peptide or protein can be produced in a bacterial or eukaryotic host organism, and then isolated from this host organism or its culture. In such a case, the method may comprise cultivation of the host cell under conditions allowing expression of the peptide or protein. It is also possible to produce a peptide or protein of the disclosure in vitro, for example, by using an in vitro translation system. When producing the peptide or protein in vivo, a nucleic acid encoding such a peptide or protein is introduced into a suitable bacterial or eukaryotic host organism using recombinant DNA technology well known in the art.

The method of production may comprise the step of folding or refolding of the peptide or protein or the target of interest. Production of pMHC molecules, for example may comprise a refolding step as for example described in Nauerth et al 2013 or Busch D H, Pilip I M, Vijh S, Pamer E G, Immunity. 1998 March; 8(3):353-62. The method of production may further comprise the step of conjugating a label as described herein to the peptide of the invention, in particular to the functionalization tag.

The present invention further relates to a protein complex comprising a peptide of the invention (including a conjugate of target of interest and peptide of the invention), a protein of the invention, and a multimerization reagent. A “multimerization reagent” as used herein may be any compound that has at least two, preferably at least three, preferably at least four binding sites for the reversible affinity tag or a molecule that has been conjugated to the functionalization tag. The protein complex is preferably a multimeric protein complex, i.e. it comprises at least two, three, or four peptides of the invention or proteins of the invention and preferably one multimerization reagent.

According to the invention, the multimerization agent may comprise a streptavidin, or avidin, or a streptavidin analog, an avidin analog that reversibly binds to said streptavidin or avidin binding peptide, or an avidin analog that reversibly binds to said biotin analog. Said said multimerization reagent may comprise the streptavidin analog having the amino acid sequence Val₄₄-Thr₄₅-Ala₄₆-Arg₄₇ (SEQ ID NO: 33) at positions 44 to 47 of the wild-type streptavidin sequence or the streptavidin analog having the amino acid sequence Ile₄₄-Gly₄₅-Ala₄₆-Arg₄₇ (SEQ ID NO: 34) at positions 44 to 47 of the wild-type streptavidin sequence. Both these muteins are described in U.S. Pat. No. 6,103,493, for example, and are commercially available under the trademark Strep-Tactin® from IBA GmbH, Gottingen, Germany. The multimerization reagent may also be an oligomer or a polymer of streptavidin or avidin or of any analog of streptavidin or avidin. The oligomer or polymer may be crosslinked by a polysaccharide. Oligomers or polymers of streptavidin or of avidin or of analogs of streptavidin or of avidin may be prepared by the introduction of carboxyl residues into a polysaccharide, e.g. dextran, essentially as described in “Noguchi, A., Takahashi, T., Yamaguchi, T., Kitamura, K., Takakura, Y., Hashida, M. & Sezaki, H. (1992). Preparation and properties of the immunoconjugate composed of anti-human colon cancer monoclonal antibody and mitomycin C dextran conjugate. Bioconjugate Chemistry 3, 132-137” in a first step. Then streptavidin or avidin or analogs thereof may be coupled via primary amino groups of internal lysine residue and/or the free N-terminus to the carboxyl groups in the dextran backbone using conventional carbodiimide chemistry in a second step. However, cross-linked oligomers or polymers of streptavidin or avidin or of any analog of streptavidin or avidin may also be obtained by crosslinking via bifunctional linkers such as glutardialdehyde or by other methods described in the literature.

The present invention also encompasses a multimerization reagent, for which binding to the peptide or protein of the invention may occur in the presence of a divalent cation. In an illustrative example, the multimerization agent may comprise two or more divalent metal atoms that are immobilized on a solid support. Such divalent metal cation may be Ni²⁺, Co²⁺, Cu²⁺ or Zn²⁺. The metal atom may be immobilized on a carrier, for example a carrier comprising NTA. The carrier may be any type of carrier, including a solid support that allows for immobilization of at least two metal cations as described herein. As an illustrative example, the carrier may comprise a magnetic bead. For example, the multimerization agent may be a nickel or cobalt-based magnetic bead as described by Tischer S et al. Int Immunol. 2012 September; 24(9):561-72.

In further illustrative examples, the multimerization reagent may comprise multimeric calmodulin as described in U.S. Pat. No. 5,985,658, for example, which may bind to a calmodulin binding peptide comprised in the peptide or protein or the complex of the invention. Alternatively, the multimerization reagent may comprise at least two, three, four, or even more antibodies that binds to an epitope that as described herein. Where the epitope tag comprises a FLAG peptide, said multimerization reagent may comprise at least two, three, four, or even more antibodies binding to the FLAG peptide, e.g. the monoclonal antibody 4E11 as described in U.S. Pat. No. 4,851,341. Similarly, the multimerization reagent may comprise at least two, three, four, or even more antibodies binding to a Myc-tag, HA-tag, VSV-G-tag, HSV-tag, and V5-tag, maltose binding protein (MBP), chitin binding protein (CBP) or thioredoxin.

In further illustrative examples, said multimerization reagent may comprise two or more glutathiones, which are capable of binding to a glutathione S-transferase (GST) comprised in the peptide, protein, or complex of the invention.

In further illustrative examples, the multimerization reagent may comprise a protein selected from the group consisting of protein A, protein G, protein a/g, and protein L, which may be capable of binding to an immunoglobulin Fc portion comprised in the peptide, protein, or complex of the invention.

The protein complex comprising the multimerization reagent may either be a reversible complex or an essentially irreversible complex. A reversible complex according to the invention means that the bond between the multimerization reagent and the peptide, protein, or complex of the invention may be disrupted. An illustrative example for a reversible complex is a complex, where the peptide, protein, or complex of the invention comprises a streptavidin or avidin binding peptide, but not a biotin, and the multimerization reagent comprises a streptavidin, or avidin, or a streptavidin analog, an avidin analog that reversibly binds to said streptavidin or avidin binding peptide. Another illustrative example for a reversible complex is a complex, wherein the peptide, protein, or complex of the invention comprises an oligohistidine tag, and the multimerization reagent comprises two or more metal atoms, nickel, cobalt, copper or zinc, such as Ni²⁺, Co²⁺, Cu²⁺ or Zn²⁺, conjugated to a carrier, such as a magnetic bead.

An irreversible complex according to the invention means that the bond between the multimerization reagent and the peptide, protein, or complex of the invention is essentially irreversible. An illustrative example for an irreversible complex is a complex, where the peptide, protein, or complex of the invention comprises a biotin, which may be conjugated to the functionalization tag, and the multimerization reagent comprises a streptavidin, or avidin, or a streptavidin analog, an avidin analog that essentially irreversibly binds biotin.

The present invention further relates to a method of determining a kinetic parameter of the binding or dissociation of the binding target of interest to a molecule said target of interest binds to, also referred herein as its “specific binding partner”. A kinetic parameter as used herein may be a dissociation constant (K_{D}), an association constant (K_{A}), a dissociation rate constant (k_{off}), or an association rate constant (kₒₙ). In this context, it is noted that the formation of complex (C) between a target of interest (T), e.g. an MHC, and its specific binding partner (L), e.g. a TCR, can be described by a two-state process noted

C [symbol] T+L.

The corresponding dissociation K_{D} constant is defined as

$${K}_{D} = \frac{[ T ] + [ L ]}{[ C ]}$$

wherein [T], [L], and [C] are the equilibrium molar concentrations of the target of interest, the specific binding partner and the respective complex at a given temperature and pressure. The association constant K_{A} is defined as the inverse of the dissociation constant K_{D}. The dissociation constant K_{D} can also be expressed as the ratio of the dissociation rate constant (k_{off}), or “off-rate” for the dissociation of the complex and the association rate constant (kₒₙ), or “on-rate” for the speed of association/formation of the complex.

K_{D}=k_{off}/kₒₙ

In the present application, the values of the thermodynamic and kinetic constants K_{d}, Kₐ, k_{ON} and k_{off} preferably refer to their determination under “standard conditions”, i.e. a temperature of 25° C. and atmospheric pressure of 1.013 bar. A preferred kinetic constant is the dissociation rate constant k_{off}.

The present invention therefore encompasses a method of determining the dissociation rate constant (k_{off}) of the target of interest and a specific binding partner. The method comprises detecting a first detectable label attached to the specific binding partner and a second detectable label attached to the target of interest. Methods of determining a dissociation rate constant are known to the person skilled in the art. In the context of the invention, determination of a dissociation rate constant may be carried out as essentially described in WO 2018/001985. FIG. 16 illustratively shows a concept of determining the dissociation rate constant.

The specific binding partner may be any entity or molecule that is or comprises a structure which the target of interest binds to. As an illustrative example, the specific binding partner may be a T cell receptor and the target of interest is a pMHC. As another illustrative example, the specific binding partner may be a cell comprising said T cell receptor, such as a T cell. According to the invention, the first detectable label may be reversibly bound to the specific binding partner, while the second detectable label may be essentially irreversibly bound to the specific binding partner. For this purpose, the specific binding partner may have been contacted with (i) a first protein complex comprising a first multimerization reagent, wherein the protein complex is a reversible complex according to the invention, the first complex comprising a first detectable label. The specific binding partner may further have been contacted with (ii) a second protein complex comprising a second multimerization reagent, wherein the second protein complex is an irreversible complex according to the invention, the second complex comprising a second detectable label. In some embodiments, the first protein complex is a complex comprising the peptide or protein of the invention comprising a streptavidin or avidin binding peptide, but not a biotin, and a multimerization reagent that comprises a streptavidin, or avidin, or a streptavidin analog, an avidin analog that reversibly binds to said streptavidin or avidin binding peptide. In some embodiments, the second protein complex is a complex comprising the peptide or protein of the invention comprising a biotin conjugated to the functionalization tag, and a multimerization reagent comprising a streptavidin, or avidin, or a streptavidin analog, an avidin analog that essentially irreversibly binds biotin.

As used herein, “reversible”, when used in the context of a monovalent binding complex, may be expressed in terms of the k_{off} rate for the binding between two binding partners, e.g. the binding between a target of interest and its specific binding partner. The k_{off} rate for reversible binding may be about 0.5×10⁻⁴ sec⁻¹ or greater, about 1×10⁻⁴ sec⁻¹ or greater, about 2×10⁻⁴ sec⁻¹ or greater, about 3×10⁻⁴ sec⁻¹ or greater, about 4×10⁻⁴ sec⁻¹ of greater, about 5×10⁻⁴ sec⁻¹ or greater, about 1×10⁻³ sec⁻¹ or greater, about 1.5×10⁻³ sec⁻¹ or greater, about 2×10⁻³ sec⁻¹ or greater, about 3×10⁻³ sec⁻¹ or greater, about 4×10⁻³ sec⁻¹, about 5×10⁻³ sec⁻¹ or greater, about 1×10⁻² sec⁻¹ or greater, or about 5×10⁻¹ sec⁻¹ or greater. The respective K_{D} value of such a reversible binding complex may be in the range of about 1×10⁻¹° M or greater, about 1×10⁻⁹M or greater, about 1×10⁻⁸M or greater, about 1×10⁻⁷M or greater, about 1×10⁻⁶M or greater, about 1×10⁻⁵M or greater, about 1×10⁻⁴M or greater, about 1×10⁻³ M or greater. In contrast thereto, “irreversible” or “essentially irreversible”, which is used synonymously and interchangeable, may also be expressed in terms of a k_{off} rate. The k_{off} rate for an (essentially) irreversible binding, e.g. between a target of interest and its specific binding partner, may be about 1×10⁻⁵ sec⁻¹ or lower, about 1×10⁻⁶ sec⁻¹ or lower, about 1×10⁻⁷ sec⁻¹ or lower, about 1×10⁻⁸ sec⁻¹ or lower, about 1×10⁻⁹ sec⁻¹ or lower, about 1×10⁻¹⁰ sec⁻¹ or lower. The respective K_{D} value of such an irreversible binding complex may be in the range of about 2×10⁻¹° M or less, about 1×10⁻¹¹M or less, or about 1×10⁻¹²M or less, about 1×10⁻¹³M or less, or about 1×10⁻¹⁴M or less. It may be in the range of 2×10⁻¹⁰ M to about 10⁻¹⁵ M. The term “about” when used herein in relation to the k_{off} rate, the kₒₙ rate or the K_{D} is meant to include an error margin of ±0.1%, ±0.2%, ±0.3%, ±0.4%, ±0.5%, ±0.7±0.9, %±1.0, %, ±1.2%, ±1.4%, ±1.6%, ±1.8%, ±2.0%, ±2.2%, ±2.4,%, ±2.6%, ±2.8%, ±3.0%, ±3.5%, ±4.0.%, ±4.5%, ±5.0%, ±6.0%, ±7.0%±, 8.0%, ±9.0%±, 10.0%, ±15.0%, or ±20.0%. However, since binding between the target of interest and its specific binding partner may be polyvalent, e.g. the specific binding partner may be a receptor that is present in multiple copies on a cell and the target of interest may be present in multimerized form, an avidity effect may have to be considered. In such a case, although the binding between a target of interest and a single specific binding partner may be reversible, the binding of an entity, such as a cell, comprising multiple specific binding partners and a reagent comprising multiple targets of interest may be essentially irreversible. For such a multivalent binding, an apparent k_{off}-value may be defined, wherein the apparent k_{off} value is the k_{off} value that may apparently be measured, if assumed that the binding is monovalent. However, if multimerization of the target of interest is reversible, the multimeric complex can be disrupted, e.g. by adding reagents that compete with the target of interest for the binding to the multimerization reagent. Hence, a reversible multimer that has two or more targets of interest bound to it may bind to an entity comprising two or more specific binding partners with a high avidity and an apparent k_{off} value which would normally indicate essentially irreversible binding as long as the target of interest is in the form of a multimer. The binding of the target of interest to the entity comprising two or more specific binding partners may still be reversible, if the multimerization itself can be reversed as described herein and the monovalent binding of the target of interest is reversible. Such reversible binding by a reversible multimer is e.g. described in U.S. Pat. No. 7,776,562, International Patent application WO 02/054065, or International Patent applications WO 2013/011011 and WO 2018/001985.

In the method of the invention, the specific binding partner may be a cell that comprises a receptor which is bound by the target of interest. Virtually any cell that has at least one common receptor molecule that can be used for k_{off} rate measurement according to the present invention. In order to achieve an avidity effect, the receptor molecule is typically present in two or more copies on the surface of the target cell. In typical embodiments the target cell is a eukaryotic or prokaryotic cell, preferably a mammalian cell. The mammalian cell may be a lymphocyte or a stem cell. Hence, the target cell may be a T cell, a T helper cell, a B cell or a natural killer cell, such as a CMV-specific a CMV-specific CD8+T-lymphocyte, a cytotoxic T-cell a, memory T-cell and a regulatory T-cell. Likewise, the at least one common (specific) receptor which defines the cell population may be any receptor for which a k_{off} rate of the binding to a given target of interest can be determined. For example, the receptor may be a receptor defining a population or subpopulation of immune cells, e.g. a population or subpopulation of T cells, T helper cells, for example, CD4⁺ T-helper cells, B cells or natural killer cells. Examples of T cells include cells such as CMV-specific CD8+T-lymphocytes, cytotoxic T cells, memory T cells and regulatory T cells (Treg). The receptor molecule may be any receptor present on the target cell. However, it is preferred that the receptor is an antigen-specific receptor, such as e.g. a T cell receptor or a B cell receptor. The receptor may preferably be a T cell receptor while the cell may preferably be a CD8+ T cell. In this context, it is noted that the term “cells” as used herein encompasses all biological entities/vesicles in which a membrane (which can also be a lipid bilayer) separates the interior from the outside environment and which comprise specific receptor molecules on the surface of the biological entity. Examples of such entities include, but are not limited to, a cell, a virus, a liposome, an organelle such as mitochondria, chloroplasts, a cell nucleus or a lysosome.

In the methods of the invention, the target of interest which specifically binds to the specific binding partner can for example be an MHC molecule. The use of an MHC molecule as a target of interest allows the characterization of a k_{off} rate of a T cell receptor of an antigen-specific subpopulation of T cells directly ex vivo.

The first multimerization reagent optionally further comprises a third detectable label. It is understood that each of the first, second, and third detectable label are preferably different from each other and can preferably be distinguished from each other. The third detectable label is preferably comprised in the first multimerization reagent of the first protein complex.

The methods of the invention may comprise a step of contacting specific binding partner with first reversible protein complex comprising a reversible multimer. The method of the present invention may further comprise the step of contacting the specific binding partner with a second irreversible protein complex comprising an irreversible multimer. The step of contacting the specific binding partner with the first protein complex may preferably be performed prior to contacting the specific binding partner with the second protein complex. Optionally, a washing step can be conducted between these two steps.

The methods of the present invention may further comprise a step of disrupting the first protein complex. It is understood that this step may be carried out after the specific binding partner has been contacted with the first protein complex as well as the second protein complex. Disruption of the first multimerization complex can be conducted by any suitable method known to the skilled person or described herein. For example, the binding can be disrupted by contacting the first protein complex with a reagent that competes with the binding of the reversible affinity tag comprised in the first protein complex to the multimerization reagent. Suitable competition reagents are described herein and depend on the type of reversible affinity tag and first multimerization reagent. As an illustrative example, where the reversible affinity tag is streptavidin or avidin binding peptide and the first multimerization reagent may be a streptavidin, avidin or analog thereof, e.g. a streptavidin mutein such as Strep-Tactin®, the competition reagent may be biotin or a biotin analog.

In a preferred embodiment, the specific binding partner is a cell, preferably a T cell, preferably a CD8+ T cell; where the target of interest binds to its T cell receptor. The targets of interest comprised in the first reversible protein complex and the second irreversible protein complex are preferably the same and are both a MHC molecule. In the first reversible protein complex, the multimerization reagent is preferably a streptavidin, avidin, or an analog thereof, preferably a streptactin that is bound to the target of interest via the reversible affinity tag, which is preferably a streptavidin binding peptide. In the second irreversible protein complex, the multimerization reagent is preferably a streptavidin, avidin, or an analog thereof, preferably a streptactin that is bound to the target of interest via a biotin that is conjugated to the functionalization tag.

A first detectable label (e.g. Alexa 488) is preferably bound to the functionalization tag of the peptide or protein of the invention comprised in the first, reversible protein complex. A second detectable (e.g. BV421) label is preferably bound to the multimerization reagent of the second irreversible protein complex. A third detectable label (e.g. APC) is preferably attached to the multimerization reagent of the first reversible protein complex. It is understood that first, second, and the optional third detectable label can be distinguished from each other. Here, biotin can be used to disrupt the first reversible protein complex.

The methods of the present invention may further comprise the step of detecting the first detectable label attached to specific binding partner and detecting the second detectable label attached to specific binding partner. Here, detecting both detectable labels may preferably be conducted after the step of disrupting the first reversible protein complex. In cases where the multimerization reagent of the first reversible protein complex comprises a third detectable label, the methods of the present invention may further comprise detecting the third detectable label. In addition, the methods of the present invention may comprise detecting (a) further detectable label(s). As an illustrative example, the specific binding partner may be additionally stained with a CD8 antibody with a further detectable label, such as e.g. eF450. The specific binding partner may also be stained with a dye that allows for discrimination between viable and dead cell. An illustrative example for such a dye is propidium iodide, which is an intercalating agent and a fluorescent molecule that is membrane impermeant and generally excluded from viable cells and which can thus be used for identifying dead cells.

It is contemplated by the invention that the detection of the detectable labels may be conducted by a flow cytometry based analysis. Flow cytometry based analysis is typically combined with optical detection to identify and classify cells and allows speed combined with high sensitivity and specificity. It allows a simultaneous multiparametric analysis of the physical and chemical characteristics of single cells flowing through an optical or electronic detection device. These specific physical and chemical characteristics may comprise the specific light scattering and/or fluorescent characteristics of each cell.

The present invention encompasses the use of two detectable labels that are directly or indirectly bound to specific binding partner. While the first detectable label is reversibly bound to the specific binding partner, binding of the second detectable label to the specific binding partner is essentially irreversible. Thus, detection of a signal of the second detectable label may be indicative for the presence of the specific binding partner or a molecule comprised in the specific binding partner to which the target of interest specifically binds. The presence or absence of the first detectable label, on the other hand, may be indicative for the non-dissociation or the dissociation of the target of interest from the specific binding partner. Here, presence of the first detectable label is indicative for the non-dissociation of the target of interest while the absence is indicative for the dissociation of the same. It is understood that the reduction of detection events of the first detectable label on a specific binding partner on which the second detectable signal is detected may be indicative for the kinetic of dissociation of the target of interest from specific binding partner. Such dissociation may follow the kinetic of an exponential decay. In analyzing the number of detection events for the first detectable label on a specific binding partner, where the second detectable label is present, the dissociation rate constant (k_{off}) for the binding of target of interest and the specific binding partner may be obtained by standard methods that are familiar to the skilled person (e.g. curve fitting).

The methods of the invention may generally allow for the determination of any kinetic parameter described herein, in particular any k_{off} values of the binding of a target of interest and its specific binding partner. However, the method is preferably applied for a binding of a receptor molecule on a target cell and a target of interest, where the k_{off} value is suspected to be within a range of about 10⁰ sec⁻¹ to about 10⁻⁴ sec⁻¹, preferably within a range of 10⁻¹ sec⁻¹ to about 10⁻³ sec⁻¹.

The methods of the invention can be carried out at any suitable temperature. Typically, the contacting of the mixture containing the specific binding partner with the first protein complex or the second protein complex or later the disruption of the first protein complex or the detection of the any one of the first detectable label, the second detectable label or the third detectable label may be carried out at such temperatures, at which substantially no activation and/or no signaling events occur, which might result in an alteration of the specific binding partner, which might be a target cell, e.g. the T cell phenotype, in case a receptor on a T cell is to be analyzed. The methods of the present invention or each individual step of the methods of the invention may thus be preferably carried out at a temperature of <15° C. or carried out at a temperature of <4° C.

The invention further encompasses that the specific binding partner may be comprised in a sample. If cases where the specific binding partner is a target cell, the sample may comprise the target cell and a plurality of other cells. The sample may comprise a population of cells (e.g. CD8+ T cells) and the target cell may be a subpopulation thereof (e.g. CD8+ T cells specific for a certain antigen).

The sample may be from any suitable source, typically all sample of a body tissue or a body fluid such as blood. The sample may thus be peripheral blood sample. In the latter case, the sample might for example, comprise a population of peripheral blood mononuclear cells (PBMC) that can be obtained by standard isolation methods such as Ficoll gradient of blood cells. The cell population comprised in the sample may however also be in purified form and might have been isolated using a reversible cell staining/isolation technology as described patent in U.S. Pat. Nos. 7,776,562, 8,298,782, International Patent application WO02/054065 or International Patent Application WO2013/011011. Alternatively, the population of cells can also be obtained by cell sorting via negative magnetic immunoadherence as described in U.S. Pat. No. 6,352,694 B1 or European Patent EP 0 700 430 B1. If an isolation method described here is used in basic research, the sample might be cells of in vitro cell culture experiments. The sample will typically have been prepared in form of a fluid, such as a solution or dispersion.

The sample may be obtained from a subject. A “subject” as used herein, refers to a human or non-human animal, generally a mammal. A subject may be a mammalian species such as a rabbit, a mouse, a rat, a guinea pig, a hamster, a dog, a cat, a pig, a cow, a goat, a sheep, a horse, a monkey, an ape or, preferably, a human. While a subject is typically a living organism, the sample may also be taken post-mortem.

The invention also encompasses a method of isolating a high-avidity T cell. This method may comprise a first step, in which the dissociation rate constant (k_{off}) of a T cell in a sample obtained from a subject is determined according to the methods of the invention. Hereby, a high-avidity T cell may be identified. A “high-avidity T cell” may be defined by the k_{off} value of the T cell receptor when binding to a given antigen, such as a pMHC which may constitute the target of interest in the peptides or proteins of the invention. The T cell may be of “high avidity”, if the k_{off} value is equal or below a given threshold value. The threshold value may depend on the purpose, for which the high-avidity T cell will be isolated for. Typically, the threshold value is in the range of about 10⁻¹ sec⁻¹ to about 10⁻³ sec⁻¹, preferably, the threshold value may be in the range of about 5×10⁻² sec⁻¹ to about 2×10⁻³ sec⁻¹, preferably about 2×10⁻² sec⁻¹ to about 5×10⁻³ sec⁻¹, preferably about 1×10⁻² sec⁻¹.

The method may then comprise a further step of isolating said T cell or population of T cells from a sample obtained from the same subject. Isolation of said T cell (population) can be performed by any method know in the art, for example by using a reversible cell staining/isolation technology as described patent in U.S. Pat. Nos. 7,776,562, 8,298,782, International Patent application WO02/054065 or International Patent Application WO2013/011011. The sample, out of which the T cell (population) is isolated in the second step may be the same sample as in the first step or may be another sample obtained from the same subject.

The present invention also relates to the use of a peptide of the invention comprising a reversible affinity tag as described herein and a functionalization tag as described herein as a peptide tag. As used therein, the peptide tag is useful when attached to a target of interest as described herein. The peptide tag may be used for versatile binding, purification, multimerization, and/or functionalization of the target of interest. If the reversible affinity tag is a sortase A recognizing sequence, it is preferred that the reversible affinity tag is N terminal of the sortase A recognizing sequence. In other words, a peptide wherein the functionalization tag is a sortase A recognizing sequence and wherein the reversible affinity tag is C terminal of the sortase A recognizing sequence may be excluded from the present invention. The peptide may be used for analyzing binding affinity or binding kinetic, such as binding affinity or binding kinetic of the target of interest conjugated to the peptide. Analysis of binding affinity or binding kinetics may comprise determination of one or more kinetic parameters according to the present disclosure. Preferably, this analysis comprises determination of a dissociation rate constant between a target of interest according to the present disclosure and its specific binding partner. The target of interest is preferably linked to the peptide according to the disclosure of the invention. The peptide may be used in any method of determining a kinetic constant as described herein.

The present invention is also characterized by following items.

Item 1. A peptide comprising (i) a reversible affinity tag (A); and (ii) a functionalization tag (F), wherein the peptide is linked to a target of interest (T), and (a) wherein the peptide and the target of interest have following configuration: T-A-F or F-A-T; or (b) wherein the peptide and the target of interest have following configuration: T-F-A or A-F-T, wherein the functionalization tag (F) is not a sortase A recognizing sequence or a tub tag.

Item 2. The peptide of any one of the preceding items, wherein the functionalization tag is a tub tag sequence, a sortase A recognizing sequence, a transglutaminase tag, a formylglycine generating enzyme recognition sequence, an avi tag, an lipoic acid ligase tag.

Item 3. The peptide of item 1 or 2, wherein the functionalization tag is a tub tag sequence or a sortase A recognizing sequence.

Item 4. The peptide of item 3, wherein the tub tag sequence is Ser-Val-Glu-Gly-Glu-Gly-Glu-Glu-Glu-Gly-Glu-Glu (SEQ ID NO: 01).

Item 5. The peptide of item 1 or 2, wherein the sortase A recognizing sequence comprises Leu-Pro-Xaa1-Thr, wherein Xaa1 is any amino acid (SEQ ID NO: 02).

Item 6. The peptide of item 5, wherein the sortase A recognizing sequence comprises Leu-Pro-Xaa1-Thr-Xaa2-Xaa3, wherein Xaa1 is any amino acid, Xaa2 is Gly or Ala, and Xaa3 is any amino acid (SEQ ID NO: 03).

Item 7. The peptide of any one of the preceding items, wherein the reversible affinity tag comprises a streptavidin or avidin binding peptide.

Item 8. The peptide of any one of the preceding items, wherein the reversible affinity tag comprises the streptavidin-binding peptide Trp-Ser-His-Pro-Gln-Phe-Glu-Lys (SEQ ID NO: 04).

Item 9. The peptide of any one of the preceding items, wherein the reversible affinity tag comprises two streptavidin-binding peptides Trp-Ser-His-Pro-Gln-Phe-Glu-Lys (SEQ ID NO: 04) separated by a linker.

Item 10. The peptide of any one of items 1 to 6, wherein the reversible affinity tag comprises an oligohistidine sequence.

Item 11. The peptide of any one of the preceding items, wherein the target of interest is a protein, a peptide, a peptidomimetic, a nucleic acid, or a polysaccharide.

Item 12. The peptide of any one of the preceding items, wherein the target of interest is a protein.

Item 13. The peptide of item 12, wherein the peptide is fused to the protein.

Item 14. The peptide of any one of the preceding items, wherein the target of interest comprises a domain of a major histocompatibility complex (MHC).

Item 15. The peptide of any one of the preceding items, wherein the target of interest comprises a polypeptide chain of a major histocompatibility complex (MHC).

Item 16. The peptide of any one of the preceding items, wherein the target of interest comprises an extracellular domain of a T cell receptor.

Item 17. The peptide of any one of the preceding items, wherein the target of interest comprises an antibody, antibody fragment, or an extracellular domain of a B cell receptor.

Item 18. The peptide of any one of items 1 to 11, wherein the target of interest comprises an aptamer.

Item 19. The peptide of any one of the preceding items, wherein the reversible affinity tag is fused to the functionalization tag.

Item 20. The peptide of any one of the preceding items, wherein the reversible affinity tag is N-terminal of the functionalization tag.

Item 21. The peptide of any one of the preceding items, wherein the target of interest is a protein or polypeptide and wherein the peptide comprising the reversible affinity tag and the functionalization tag are fused C-terminal of target of interest.

Item 22. The peptide of any one of the preceding items, wherein a label is conjugated to the functionalization tag.

Item 23. The peptide of item 22, wherein the label is biotin or a biotin analog.

Item 24. The peptide of item 22, wherein the label is a detectable label.

Item 25. The peptide of item 24, wherein the detectable label is a fluorescent label.

Item 26. The peptide of item 24, wherein the detectable label is a nucleic acid.

Item 27. The peptide of item 22, wherein the label is a toxin.

Item 28. The peptide of any one of the preceding items having the configuration T-A-F, wherein the functionalization tag is a sortase A recognizing sequence, comprising a further affinity tag that is C-terminal of the sortase A sequence.

Item 29. The peptide of item 28, wherein the further affinity tag is an oligohistidine tag.

Item 30. A protein comprising the peptide of any one of the preceding items.

Item 31. The protein of item 30 comprising a peptide major histocompatibility complex (pMHC).

Item 32. A nucleic acid encoding the peptide of any one of items 1 to 29 or the protein of item 30 or 31.

Item 33. A vector comprising a nucleic acid of item 32.

Item 34. A host cell comprising the nucleic acid of item 32 or the vector of item 33.

Item 35. A method of producing a peptide of any one of items 1 to 29 or a protein of item 30 or 31, comprising cultivation of the host cell of item 34 under conditions allowing expression of the peptide or the protein.

Item 36. The method of item 35 further comprising the step of conjugating a label as defined in any one of items 22 to 27 to the peptide.

Item 37. A protein complex comprising a peptide of any one of items 1 to 29 or a protein of item 30 or 31 and a multimerization reagent.

Item 38. The complex of item 37 comprising a peptide of any one of items 7 to 9, 11 to 22 and 24 to 27 comprising a streptavidin or avidin binding peptide as reversible affinity tag, wherein the multimerization reagent is a streptavidin, avidin, streptavidin analog, or avidin analog that essentially reversibly binds to the streptavidin or avidin binding peptide.

Item 39. The complex of item 37 comprising a peptide of any one of items 10 to 27 comprising an oligohistidine tag as reversible affinity tag, wherein the multimerization reagent comprises two or more nickel or cobalt atoms that essentially reversibly binds to the oligohistidine tag.

Item 40. The complex of item 37 comprising a peptide of item 23 and wherein the multimerization reagent is a streptavidin, avidin, streptavidin analog, or avidin analog that essentially irreversibly binds to a biotin or a biotin analog comprised in the peptide.

Item 41. A method of determining the dissociation rate constant (k_{off}) of a specific binding partner and a target of interest, comprising detecting a first detectable label attached to the specific binding partner and a second detectable label attached to the target of interest, wherein the specific binding partner has been contacted with (i) a first protein complex of item 38 or 39 comprising at least one peptide comprising a first detectable label and a first multimerization reagent, and (ii) a second protein complex of item 40 comprising at least one peptide and a second multimerization reagent, wherein the at least one peptide of the second complex or the second multimerization reagent comprises a second detectable label that can be distinguished from the first detectable label.

Item 42. The method of item 41 wherein the specific binding partner comprises a T cell receptor and the target of interest is a peptide major histocompatibility complex (pMHC).

Item 43. The method of item 41 or 42, wherein the specific binding partner is a cell comprising a T cell receptor.

Item 44. The method of any one of items 41 to 43, wherein the first multimerization reagent comprises a third detectable label that can be distinguished from the first detectable label and the second detectable label.

Item 45. The method of any one of items 41 to 44 comprising the steps of (a) contacting said specific binding partner with the first protein complex; and (b) contacting said specific binding partner with the second protein complex.

Item 46. The method of any one of items 41 to 45 comprising the step of (c) disrupting the first protein complex.

Item 47. The method of item 46, wherein disruption of the first protein complex is effected by addition of a competition reagent.

Item 48. The method of any one of items 41 to 47 comprising the step of (d) detecting the first detectable label attached to the specific binding partner and detecting the second detectable label attached to the specific binding partner.

Item 49. The method of any one of items 41 to 48, wherein the detection of the first detectable label and the second detectable label is by flow cytometry.

Item 50. A method of isolating a high-avidity T cell comprising (a) determining the dissociation rate constant (k_{off}) of a T cell receptor on a T cell in a sample obtained from a subject using the method of any one of items 41 to 49, (b) isolating said T cell from a sample obtained from said subject.

Item 51. Use of a peptide comprising (i) a reversible affinity tag; (ii) a functionalization tag; as a peptide tag.

Item 52. The use of item 51, wherein the peptide is conjugated to a target of interest.

Item 53. The use of item 52 for analyzing binding affinity or binding kinetics of the target of interest.

Item 54. The use of item 53 for determination of a dissociation constant (K_{D}), an association constant (K_{A}), a dissociation rate constant (k_{off}), or an association rate constant (kₒₙ) between a target of interest that is linked to the peptide and a specific binding partner of the target of interest.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “about” or “approximately” as used herein means within 20%, preferably within 10%, and more preferably within 5% of a given value or range. It includes, however, also the concrete number, e.g., about 20 includes 20.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent applications, scientific publications, manufacturer's specifications, instructions, etc.) are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

### EXAMPLES

### Example 1

We performed proof-of-concept experiments to test if we could use the strategy of combining a site-specific functionalization tag with a reversible multimerization tag to conjugate biotin or dyes to Strep- and Tub-tagged FLEXamers. We generated two different FLEXamers for the HLA class I heavy chains B*07:02 and B*08:01, which present Cytomegalovirus (CMV) pp65 and IE1, respectively (FIG. 2a; FIG. 9). Enzymatic activation of the common precursor FLEXamer and subsequent conjugation with biotin (Bio), Atto488 (A488) or sulfo-cyanine5 (sCy5) was highly efficient (FIG. 2b).

### Example 2

We then tested whether the non-reversible biotinylated FLEXamer, the reversible dye-conjugated FLEXamer and their reversible FLEXamer precursor could fulfill their distinct functions. Biotinylated FLEXamers stained B*07:02/pp65₄₁₇₋₄₂₆-specific T cells from peripheral blood of a CMV-seropositive donor with high sensitivity and no difference to conventionally biotinylated tetramers (FIG. 3). An irrelevant epitope/MHC combination (A*02:01/Her2neu₃₆₉₋₃₇₇) served as control for un-specific staining (FIG. 3). As expected, binding could not be reversed upon addition of D-biotin (FIG. 10).

### Example 3

To test whether the functionalization-tag interferes with reversibility, we stained and flow-sorted B*07:02/pp65₄₁₇₋₄₂₆-specific CD8⁺ T cells from peripheral blood of a CMV-seropositive donor either with conventional Streptamers or FLEXamers (FIG. 4). FLEXamers could stain B*07:02/pp65₄₁₇₋₄₂₆-specific T cells and allowed high purity flow cytometric sorting like conventional Streptamers (FIG. 4b). Upon addition of D-biotin, the pMHC label could be detached. Complete removal of pMHC monomers from the cells is demonstrated by the inability to re-stain the cells by solely adding the Strep-Tactin backbone, whereas addition of the multimerized FLEXamer resulted in efficient re-staining (FIG. 4b). The additional functionalization tag, here a Tub-tag, therefore does not interfere with the reversible binding of the FLEXamer reagent.

### Example 4

Conjugation of dyes to Streptamer pMHCs allows direct tracing of pMHC monomer dissociation kinetics on the single-cell level after addition of D-biotin, in order to measure TCR:pMHC koff-rates for TCR structural avidity estimation (Nauerth, M. et al. Sci. Transl. Med. 5, 192ra87 (2013); Nauerth, M. et al. Cytom. Part A 89, 816-825 (2016)) (FIG. 5). When a B*07:02/pp65417-426 T cell line was stained with dye-conjugated Streptamers or FLEXamers, the dye-conjugated pMHC monomers showed monomeric pMHC dissociation after initial dye dequenching as previously described (Nauerth, M. et al. Sci. Transl. Med. 5, 192ra87 (2013)) (FIG. 5b). Fitting of exponential decay curves yielded defined k_{off}-rates for the TCR:pMHC interaction that were identical for dye-conjugated Streptamers and FLEXamers (FIG. 5d).

Using a double staining with a non-reversible biotinylated pMHC multimer and a reversible dye-conjugated pMHC Streptamer, dissociation kinetics can be tracked without previous purification on a flow cytometer through continuous gating on the non-reversible pMHC multimer⁺ T cell population (Nauerth, M. et al. Cytom. Part A 89, 816-825 (2016)). This emphasizes that not only the versatile nature of the different pMHC constructs themselves, but also their combinatorial usage has made them become indispensable tools for in-depth T cell characterization. We stained a heterogeneous B*07:02/pp65₄₁₇₋₄₂₆-specific T cell population directly ex vivo with both non-reversible pMHC conjugated to biotin and reversible pMHC conjugated to A488 (FIG. 5c). Non-reversible pMHC multimers allowed continuous gating on the antigen-specific T cell population after the addition of D-biotin (FIG. 11a), while the reversible fluorophore-conjugated pMHC monomers dissociated over time (FIG. 5c). The heterogeneous T cell populations specific for B*07:02/pp65₄₁₇₋₄₂₆ entailed two kinetics (FIG. 5c; FIG. 11c). We retrieved cell lines from both kinetics and stained them with dye-conjugated Streptamers and FLEXamers. We again obtained highly comparable dissociations resembling the dissociation of the parental heterogeneous T cell population (FIG. 11c).

### Example 5

Next, we set out to test the general applicability of our double-tag approach. Therefore, in addition to an HLA-A*02:01 heavy chain Tub-tagged construct, we generated FLEXamers with both a Strep-tag and a sortase A (SrtA) recognition site to allow versatile protein conjugation (Popp, M. W. et al. Nat. Chem. Biol. 3, 707-8 (2007)), as well as a His-tag to allow for fast and efficient protein purification after transpeptidation (FIG. 6a; Supplementary FIG. 5). We stained PBMCs with a transgenic TCR specific for A*02:01/pp65₄₉₅₋₅₀₃ with Tub-tag- or SrtA-biotinylated tetramers (FIG. 6b) and also tested reversibility of the SrtA-tag carrying FLEXamer precusor (FIG. 6c). Furthermore, Tub- or SrtA-tag dye-conjugated reversible FLEXamers were tested for characterization of TCR:pMHC k_{off}-rates (FIG. 6d-e). Each time, SrtA FLEXamers, Tub-tag FLEXamers, and their biotin- or dye-conjugated downstream pMHC products performed equally well (FIG. 6b-e) independent of the respective functionalization strategy. As compared to SrtA-tag, Tub-tag-mediated pMHC functionalization is overall even more efficient which is accompanied with significantly reduced educt consumption. This shows that not a specific sequence, but the combination of a reversible affinity tag and a functionalization tag in one double-tag enables fast and efficient generation of any pMHC construct with fully preserved functionality. Tub-tag technology to generate FLEXamers may however be preferred.

### Example 6

Encouraged by the simple generation process of different pMHC multimer reagents from a single double-tagged FLEXamer precursor, we generated FLEXamers also for other epitope-HLA combinations. For B*08:01 presenting IE1_{199-207K} we validated the equal functionality of non-reversible, reversible and fluorophore-conjugated FLEXamers (FIG. 13). To even further extend the set of available FLEXamers we folded in total 26 FLEXamers covering nine HLA class I heavy chains as well as the murine heavy chain H2-K^{b} (FIG. 7a). The conjugation efficacy with fluorophore or biotin was consistently high for all FLEXamers (FIG. 7b). Due to the skewed frequency distribution of HLA class I alleles, the nine human HLA heavy chains together cover 76.5% of the EURCAU population (FIG. 7c-d), and also entail two allotypes (A*24:02 and A*11:01) which are highly prevalent in Asian populations. This set of FLEXamers can serve as precursors for any kind of pMHC reagent. the set of available FLEXamers we folded in total 26 FLEXamers covering nine HLA class I heavy chains as well as the murine heavy chain H2-K^{b} (FIG. 7a). The conjugation efficacy with fluorophore or biotin was consistently high for all FLEXamers (FIG. 7b). Due to the skewed frequency distribution of HLA class I alleles, the nine human HLA heavy chains together cover 76.5% of the EURCAU population (FIG. 7c-d), and also entail two allotypes (A*24:02 and A*11:01) which are highly prevalent in Asian populations. This set of FLEXamers can serve as precursors for any kind of pMHC reagent.

The heterogeneity of infectious agents and cancers is met by the adaptive immune system's ability to present and recognize many different targets. The total epitope repertoire has been estimated to be in between 10⁶ and 10¹¹in mice (Cohn, M. Immunol. Res. 64, 795-803 (2016)) and is likely similarly, if not even more diverse in humans. More than 13.000 HLA class I alleles have now been described for humans (Robinson, J. et al. Nucleic Acids Res. 43, D423-31 (2015)), and the total human TCR repertoire encompasses more than 10⁸ unique clonotypes (Qi, Q. et al. Proc. Natl. Acad. Sci. 111, 13139-13144 (2014)). Customized monitoring of antigen-specific immune responses and individualized immunotherapy therefore require streamlined methods that allow flexible adaptation for each patient and disease in terms of first, target-specific epitopes, and second, patient-specific HLAs. The versatile applicability of pMHC multimer reagents—for T cell identification, traceless isolation or TCR avidity measurement—makes them particularly valuable tools for the investigation and therapeutic usage of T cells (Davis, M. M., Altman, J. D. & Newell, E. W. Nat. Rev. Immunol. 11, 551-558 (2011)), but consequently adds even a third level of complexity.

In order to be compatible with the extreme diversity of epitopes, UV exchange (Toebes, M. et al. Nat. Med. 12, 246-51 (2006)) or di-peptide (Saini, S. K. et al. Proc. Natl. Acad. Sci. U.S.A 112, 202-7 (2015)) technologies have been developed that can be used to load HLA class I with any epitope of interest. In addition, combinatorial pMHC staining (Hadrup, S. R. et al. Nat. Methods 6, 520-6 (2009); Newell, E. W. et al. Nat. Methods 6, 497-499 (2009)) and DNA barcoding (Bentzen, A. K. et al. Nat. Biotechnol. (2016). doi:10.1038/nbt.3662) have massively enhanced throughput of screening antigen-specific T cell populations and their respective TCR repertoires. Despite this progress, however, difficulties to generate distinct pMHC multimer reagents appropriate for each individual setting in a fast and reliable manner remain a significant challenge to personalized cell therapy and T cell-based diagnostics. Importantly, for broad applicability of pMHC multimer reagents, the production process of different kinds of pMHC multimer reagents needs to be feasible also for many different HLA class I heavy chains.

FLEXamers combine the provision of versatility through distinct pMHC constructs with a simple generation process from a single precursor protein (FIG. 1). FLEXamers are as functional as conventionally generated pMHC based reagents, while being produced in a faster and more standardized manner. Core feature of FLEXamers is a novel double-tag that allows reversible multimerization as well as functionalization with any probe of interest. In this study we provided proof-of-concept to generate biotinylated tetramers, reversible Streptamers or reversible dye-conjugated pMHC multimer reagents suitable for k_{off}-rate measurements from a common FLEXamer precursor protein. Notably, the use of FLEXamers is not limited to these specific applications as the functionalization-tag also allows conjugation e.g. of DNA oligonucleotide sequences, toxins (FIG. 2a) and many more entities. Furthermore, FLEXamers can be easily combined with epitope exchange technologies (Saini, S. K. et al. Proc. Natl. Acad. Sci. U.S.A 112, 202-7 (2015); Rodenko, B. et al. Nat. Protoc. 1, 1120-1132 (2006)).

The effort and costs needed to generate distinct pMHC multimer reagents for each application has so far been a key obstacle for laboratories to fully exploit the versatility of pMHC based reagents. Double-tagged pMHC FLEXamers can be easily generated and applied. Functionalization of double-tagged pMHCs is not confined to Tub-tag technology and can also be achieved e.g. via SrtA. Notable advantages of Tub-tag technology are mild reaction conditions with simultaneously high conjugation efficiencies using click chemistry. We used Tub-tag technology to generate a set of versatile pMHC FLEXamers for 9 different human HLAs as well as murine H2-K^{b}.

Multivalent binding can serve as an ‘on-switch’ to stabilize otherwise transient binding of weak interaction partners. In turn, receptor-ligand binding can be switched off via disruption of the multimeric complex, which requires that the multimerization is reversible. Versatile functionalization thereby allows further stabilization of the interaction, or tracking via fluorescent dyes. The field of T cell immunology has made extensive use of this trick through multimerization of pMHC monomers. Our double-tag approach enables universal generation of different pMHC constructs, but also constitutes a flexible tool for investigation of transient protein-protein interactions in general.

### Example 7

FIG. 14 shows proof of concept that MMAF functionalized Tub-tagged Streptamers allow targeted killing of Ag-specific CD8 T cells. Specificity is demonstrated by the fact, that 0.25 ug MMAF coupled A2/pp65₄₉₅₋₅₀₃ Streptamers reduce the Ag-specific CD8 T cell population down to 15.2% in comparison to 64.5% when cells were cultured with un-functionalized A2/pp65₄₉₅₋₅₀₃ Streptamers or 60.7% when cultured with the irrelevant A1/pp65₍₃₆₃₋₃₇₃₎ Streptamer. Increasing the amount of toxin armed Streptamers to 0.5 ug reduces the population of Ag-specific CD8 T cells down to 4.9%. However, 0.5 ug un-functionalized Streptamer does also reduce the size of the Ag-specific population about 20% to 43.7%. This is explained by activation induced cell death mediated by the Streptamers. Increasing the amount of irrelevant toxin armed Al/pp65₍₃₆₃₋₃₇₃₎Streptamer does not affect the size of the Ag-specific CD8 T cell population, as the cells cannot bind to the Streptamer.

### Example 8

FIG. 15 shows proof of concept that single-stranded oligonucleotides can be conjugated to double tagged MHC heavy chains. FLEXamer A2/pp65₄₉₅₋₅₀₃ was conjugated with DBCO-PEG₄-Biotin and DBCO-PEG₄-Atto488 as control reactions. Efficient conjugation can be observed by SDS-PAGE analysis and Coomassie staining but no conjugation is observed when omitting either azido-tyrosine (wo Y-N3) or the DBCO-containing click reagent (contr). Further oligonucleotide) (Oligo1) was conjugated under the same conditions using a 4× molar excess over A2/pp65₄₉₅₋₅₀₃. Conjugation was analyzed by SDS-PAGE and Coomassie staining, revealing an additional band at approximately 60 kDa representing the MHC heavychain-Oligo conjugate. Two more FLEXamers, A2/E6 and A2/E7, were labeled with Oligo) and Oligo2 under the same conditions using an 8×molar excess yielding FLEXamer-Oligo conjugates.

### Example 9

Double-Tagging of the Extracellular Domain of Chimeric Antigen Receptors with Strep- and Tub-Tag for Dye Conjugation

Chimeric antigen receptor (CAR) transgenic T cells emerge as powerful tool to fight B cell malignancies. In order to measure the binding strength of αCD19 CAR expressing T cells to their CD19 positive B cell target, we cloned the Strep- and Tub-tag to the extracellular domain (ECD) of a αCD19 CAR (SEQ ID NO: 42). After recombinant protein expression, the CAR was functionalized using FLEXamer technique and subsequently multimerized on APC labeled StrepTactin as done previously for their pMHC multimer counterparts. The resulting fluorescently labeled reversible CAR-multimers were used to measure the dissociation kinetic of the CAR on living B cells by flow based k_{off}-rate measurement (FIG. 17). After addition of D-biotin, the StrepTactin APC backbone dissociates quickly (FIG. 17 b left) leaving monomeric, slowly dissociating fluorescently labeled CAR on the B cells surface (FIG. 17 b right). Decay in fluorescence intensity was used to fit a one-phase exponential decay curve (FIG. 17 c) to determine the k_{off}-rate of the CAR (FIG. 17 d).

Experimental Procedure

TTL Expression and Purification

TTL was expressed and purified according to a published protocol (Schumacher, 2015) as follows. The TTL (Canis lupus) coding sequence was amplified from a mammalian expression vector (S. Zink, L. Grosse, A.2012), cloned into a pET28-SUM03 (EMBL-Heidelberg, Protein Expression Facility) and expressed in E. coli BL21(DE3) as Sumo-TTL fusion protein with an N-terminal His-Tag. Cells were induced with 0.5 mM IPTG and incubated at 18° C. for 18 h. Lysis was performed in presence of Lysozyme (100 μg/ml), DNAse (25 μg/ml) and PMSF (2 mM) followed by sonification (Branson® Sonifier; 5 times 7×8 sec, 40% amplitude) and debris centrifugation at 20.000 g for 30 min. His-Sumo-TTL was purified using a 5 ml His-Trap (GE Healthcare). Purified protein was desalted on a PD10 column (GE Healthcare); buffer was exchanged to MES/K pH 7.0 (20 mM MES, 100 mM KCl, 10 mM MgCl₂) supplemented with 50 mM L-glutamate, 50 mM L-arginine. Protein aliquots were shock-frozen and stored at −80° C.

Cloning of Tub- and Sortase-Tag into Streptamer Expression Vector

pET3a expression vectors containing the coding sequence of strep-tagged HLA allotypes, including murine H2Kb, served as parental plasmid to insert the Tub-tag or Sortase A tag sequence seamless downstream of strep-tag. All insertions were performed using the Q5® Site-Directed Mutagenesis Kit (New England BioLabs, Frankfurt, Germany) following manufactures protocol. Insertion primers (Sigma Aldrich, Taufkirchen, Germany) contained 18 bp plasmid binding sequence flanking the integration site and encoded ½ of the Tub- or SrtA-tag sequence.

Generation of pMHC Monomers

All pMHC monomers described in this report, including the double-tagged pMHC molecules, were generated as published previously (Busch, D. H. et al. J. Exp. Med. 188, 61-70 (1998); Knabel, M. et al. Nat. Med. 8, 631-7 (2002)). In brief, recombinatly expressed and purified human as well as murine MHC heavy chain and β2 microglobuline were denatured in urea and subsequently refolded into the heterotrimeric pMHC complex in presence of an excess of peptide (synthetic peptides purchased by Peptide & Elephants, Potsdam, Germany). Correctly folded pMHC monomers were purified using size exclusion chromatography, concentrated and stored at −80° C. or in liquid nitrogen. All conventional dye-conjugated Streptamers used for k_{off} rate measurements were generated by maleimid chemistry using a solvent exposed artificial cysteine residue as described (Nauerth et al Sci Tranl Med 2013).

TTL Reaction on Tub-Tagged FLEXamers

TTL catalyzed ligation of 3-azido-L-tyrosine (Watanabe Chemical Industries LTD, Hiroshima) to Tub-tagged FLEXamers was performed in 25-100 uL consisting of 20 μM FLEXamer, 5 μM TTL and 1 mM 3-azido-L-tyrosine in TTL-reaction buffer (20 mM MES, 100 mM KCl, 10 mM MgCl₂, 2.5 mM ATP and 5 mM reduced glutathione) at 25° C. for 3 h followed by buffer exchange to 20 mM Tris.HCl, 50 mM NaCl pH 8.0 by size exclusion chromatography (Zeba Spin desalting columns, 7K MWCO, Thermo Scientific). Azido-FLEXamers were stored at 4° C. or directly used for click-functionalization.

Click-Functionalization of Azido-FLEXamers

Azido-FLEXamers were functionalized by incubation of 20 μM Azido-FLEXmer with either 400 μM DBCO-PEG₄-Biotin, 400 μM DBCO-sulfoCy5 or 200 μM DBCO-PEG₄-Atto488 (Jena Bioscience, Jena Germany) for 18 h at 16° C. followed by buffer exchange to 20 mM Tris, 50 mM NaCl pH 8.0 and storage at −80° C. Conjugation was analyzed by reducing SDS-PAGE and Coomassie staining. In addition, biotinylated FLEXamers were plotted on a nitrocellulose membrane, stained with a streptavidin-Alexa Fluor 594 (Dianova, Germany) conjugate and detected on an Amersham Imager 600 system. In-gel fluorescence of fluorophore labeled FLEXamers was directly detected using the same instrumentation.

Functionalization of SrtA-Tagged FLEXamers

10 μM SrtA-tagged Flexamer was incubated with 1 mM Gly₅-FITC or 1 mM Gly₅-biotin peptide and 30 μM SrtA (kindly provided as purified enzyme derived from S. aureus by Dr. Hannelore Meyer; Technical University of Munich) in 20 mM HEPES, 5 mM CaCl₂, pH 7.5 at 25° C. for 18 h. Ni-NTA sepharose bead based pulldown in PBS, 20 mM imidazole, pH 8.0 at 4° C. for 30 min was used to remove His-tagged SrtA and SrtA-tagged FLEXamer educts still carrying the His-tag. Purified functionalized SrtA-tagged FLEXamers were buffer exchange after functionalization to 20 mM Tris, 50 mM NaCl, pH 8.0. Conjugation and purification were analyzed by SDS-PAGE followed by detection of in-gel fluorescence and Coomassie staining.

CMV Reactive Primary T Cells and T Cell Lines

CMV reactive T cell lines were generated and cultured as described previously (Nauerth et al Sci Tranl Med 2013). Primary T cells reactive for CMV were derived from healthy CMV seropositive donors. Written informed consent was obtained from the donors, and usage of the blood samples was approved according to national law by the local Institutional Review Board (Ethikkommission der Medizinischen Fakultät der Technischen Universitat München). Blood was diluted 1:1 with sterile PBS and PBMCs isolated by density gradient centrifugation using Leucosep tubes (Greiner bio-one, Heidelberg, Germany) following manufacturers protocol.

pMHC Multimer and Antibody Staining

All reversible pMHC monomers (with and w/o dye) were multimerized on Streptacin APC or Streptactin PE (IBA, Gottingen, Germany) by incubating 1 μg reversible pMHC monomer and 1 μl Streptactin APC or PE in a total volume of 50 μl FACS buffer for 30 min on ice in dark. Conventionally biotinylated pMHC monomers for generation for non-reversible multimers were generated as described (Busch et al. J. Immunol. 160, 4441-8 (1998)). Subsequently, all biotin functionalized pMHC monomers described in this report, were multimerized by, incubation of 1 μg biotinylated pMHC monomers with 1.25 μg Streptavidin BV421, Streptavidin PE or Streptavidin APC (Biolegend) in a total volume of 50 μl FACS buffer for 30 min on ice in dark. For k_{off} rate measurements, up to 5×10⁶ cells were incubated with dye-conjugated reversible pMHC multimers for 45 min on ice in dark. Antibodies staining (CD8 eF450 eBioscience) was added after 25 min and incubated for additional 20 min. If combinatorial staining with non-reversible pMHC multimers were performed, cells were washed and incubated for 10 min with non-reversible pMHC multimers on ice in dark. For live/dead discrimination cells were washed in propidium iodide solution. When solely performing pMHC multimer staining with a combination of non-reversible pMHC multimers, staining was incubated for 30 min on ice in dark. We routinely stain the pMHC multimer conjugated to the smaller dye first. After incubation, cells are washed and stained with the second pMHC multimer for 30 min. Antibody staining was added after 10 min and incubated for additional 20 min. When cells were stained with reversible pMHC multimers for traceless cell isolation, samples were incubated for 45 min with the multimer regent. After 25 min, antibody staining was added and incubated for additional 20 min. All FACS data was analyzed with FlowJo software (FlowJo, LLC, Ashland).

FACS Analysis and Flow Sorting

Acquisition of FACS samples was done on a CyAn ADP Px9 color flow cytometer (Beckman Coulter, Miami, USA). Flow sorting was conducted on a MoFlo legacy (Beckman Coulter). K_{off} measurements were performed as described (Nauerth et al Cytometry 2016). In brief, samples were transferred into precooled FACS tubes containing a total volume of 1 ml FACS buffer and placed into a Peltier cooler (qutools GmbH, Munich, Germany) set to 5.5° C. After 30 sec acquisition, 1 ml of cold 2 mM D-biotin was added into the ongoing measurement. Dissociation kinetics are measured for 15 min. For analysis of k_{off} data, fluorescence data of antigen-specific were are exported from FlowJo to PRISM (GraphPad Software, San Diego, USA). Half-lives were determined by fitting a one-phase exponential decay curve.

Cloning, Expression, and Preparation of αCD19 CAR Multimers and k_{off}-Rate Measurement

The extracellular domain (ECD) of an αCD19 CAR was recombinantly expressed in E. coli and purified by size exclusion and Strep-tag affinity chromatography. Subsequently, the αCD19 CAR ECD was fluorescently labeled using FLEXamer technology as described. 0.2 μg fluorescently labeled αCD19 CAR ECD was multimerized on 1 μl StrepTactin APC in a total of 50 μl FACS buffer for 1 h on RT. Resulting fluorescently labeled reversible CAR multimers were used to stain PBMCs for 45 min on 4° C. αCD20 mAb was added for the last 20 min of the staining. Flow cytometry based k_{off}-rates measurement was performed on Beckman Coulter Cytoflex for 30 min on RT. After the first 30 sec, D-biotin was added to a final concentration of 1 mM to disrupt the CAR multimer.

The invention illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1.-54. (canceled)

55. A peptide comprising
(i) a reversible affinity tag (A); and
(ii) a functionalization tag (F),
wherein the peptide is linked to a target of interest (T), and
(a) wherein the peptide and the target of interest have following configuration:
T-A-F or F-A-T; or
(b) wherein the peptide and the target of interest have following configuration:
T-F-A or A-F-T,
wherein the functionalization tag (F) is not a sortase A recognizing sequence or a tub tag.

56. The peptide of claim 55, wherein the functionalization tag is a tub tag sequence, a sortase A recognizing sequence, a transglutaminase tag, a formylglycine generating enzyme recognition sequence, an avi tag, or a lipoic acid ligase tag.
streptavidin or avidin binding peptide

57. The peptide of claim 55, wherein the reversible affinity tag comprises a streptavidin or avidin binding peptide, or
an oligohistidine sequence.
peptidomimetic, a nucleic acid, or a polysaccharide.

58. The peptide of claim 55 wherein the target of interest is a protein, a peptide, a peptidomimetic, a nucleic acid, or a polysaccharide.

59. The peptide of claim 55, wherein the target of interest is a protein.

60. The peptide of claim 59, wherein the peptide is fused to the protein.

61. The peptide of claim 55, wherein the target of interest comprises
(i) a domain of a major histocompatibility complex (MHC);
(ii) a polypeptide chain of a major histocompatibility complex (MHC);
(iii) an extracellular domain of a T cell receptor;
(iv) an antibody, antibody fragment, or an extracellular domain of a B cell receptor; or
(v) an aptamer.

62. The peptide of claim 55, wherein the target of interest is a protein or polypeptide and wherein the peptide comprising the reversible affinity tag and the functionalization tag are fused C-terminal of target of interest.

63. The peptide of claim 55, wherein a label is conjugated to the functionalization tag.

64. The peptide of claim 63, wherein the label is
(i) biotin or a biotin analog;
(ii) a detectable label;
(iii) a fluorescent label;
(iv) a nucleic acid; or
(v) a toxin.

65. The peptide of claim 55 having the configuration T-A-F, wherein the functionalization tag is a sortase A recognizing sequence, comprising a further affinity tag that is C-terminal of the sortase A sequence.

66. A protein comprising the peptide of claim 55.

67. The protein of claim 66 comprising a peptide major histocompatibility complex (pMHC).

68. A nucleic acid encoding the peptide of claim 55 or a protein comprising said peptide.

69. A vector comprising a nucleic acid of claim 68.

70. A host cell comprising the nucleic acid of claim 68 or a vector comprising said nucleic acid.

71. A method of producing a peptide of claim 55 or a protein comprising said peptide, comprising cultivation of a host cell comprising a nucleic acid encoding said peptide or protein or a vector comprising said nucleic acid under conditions allowing expression of said peptide or said protein.

72. A protein complex comprising a peptide of claim 55 or a protein comprising said peptide and a multimerization reagent.

73. A method of determining the dissociation rate constant (k_{off}) of a specific binding partner and a target of interest, comprising detecting a first detectable label attached to the specific binding partner and a second detectable label attached to the target of interest, wherein the specific binding partner has been contacted with
(i) a first protein complex of claim 72 comprising a streptavidin or avidin binding peptide as reversible affinity tag, wherein the multimerization reagent is a streptavidin, avidin, streptavidin analog, or avidin analog that essentially reversibly binds to the streptavidin or avidin binding peptide or a first protein complex of claim 72 comprising an oligohistidine tag as reversible affinity tag, wherein the multimerization reagent comprises two or more nickel or cobalt atoms that essentially reversibly binds to the oligohistidine tag, wherein the first protein complex comprises at least one peptide comprising a first detectable label and a first multimerization reagent, and
(ii) a second protein complex of claim 72, wherein a biotin is conjugated to the functionalization tag of the peptide, and wherein the multimerization reagent is a streptavidin, avidin, streptavidin analog, or avidin analog that essentially irreversibly binds to a biotin or a biotin analog comprised in the peptide, wherein the second protein complex comprises at least one peptide and a second multimerization reagent,
wherein the at least one peptide of the second complex or the second multimerization reagent comprises a second detectable label that can be distinguished from the first detectable label.

74. A method of isolating a high-avidity T cell comprising
(a) determining the dissociation rate constant (k_{off}) of a T cell receptor on a T cell in a sample obtained from a subject using the method of claim 73,
(b) isolating said T cell from a sample obtained from said subject.
